(19) United States
(12) Patent Application Publication
(10) Pub. No.: US 2007/0036776 A1
(43) Pub. Date: Feb. 15, 2007

(54) Bacterial signaling molecules that down-regulate pathogenic bacterial virulence properties

(76) Inventors: Gregor Reid, London (CA); Peter Cadieux, London (CA); John McCormick, London (CA); Estelle Devillard, Champleey (FR)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/363,915

(22) Filed: Feb. 28, 2006

Related U.S. Application Data
(63) Continuation-in-part of application No. PCT/IB04/03126, filed on Aug. 27, 2004.

(51) Int. Cl. A61K 35/74

(52) U.S. Cl. 424/93.45; 424/780

(57) ABSTRACT

This invention relates to composition and methods of employing said composition for treating or preventing microbial associated infections and diseases. More particularly the present invention relates to bacterial proteins, peptides and amino acids which are by-products of bacteria, in particular Lactobacillus and more specifically Lactobacillus strains GR-1 and RC-14, in compositions that can treat and prevent microbial-associated infections and diseases, by altering, for example, down-regulating, virulence properties of pathogenic organisms.

### CROSS REFERENCE TO RELATED APPLICATIONS

This is a continuation-in-part of International Application PCT/IB2004/003126, with an international filing date of Aug. 27, 2004, which claims the benefit of U.S. Provisional Application No. 60/498,960 filed Aug. 29, 2003.

### FIELD OF THE INVENTION

This invention relates to bacterial proteins, peptides and amino acids which are by-products of Lactobacillus, preferably, Lactobacillus GR-1 and/or Lactobacillus reuteri RC-14, and which are not hydrogen peroxide, lactic acid, antimicrobial pheromones, mucin-inducer, biosurfactants or bacteriocins previously known in the art, in compositions that can treat and prevent infections and diseases, by altering (e.g., down-regulating) virulence properties of pathogenic organisms and/or enhancing host defenses.

### BACKGROUND OF THE INVENTION

Microorganisms still represent one of the top three causes of death amongst humans and animals. The offending organisms can be bacteria, fungi, protozoa, viruses and other forms, collectively termed as pathogens (or organisms which behave like pathogens under certain situations, such as host defense compromise). Pathogens use many different factors to cause disease, including adhesins which colonize tissues and surfaces, toxins, slime and other capsular substances, antibiotic-resistance genes, immune modifiers and substances which help escape immune responses, for example.

The primary exogenous mechanism to eradicate offending pathogens is antimicrobial agents, such as antibiotics. However, these agents are often ineffective due to resistance of the offending organisms, inability to eradicate biofilms and poor penetration at the tissue or biomaterial interface.

There are a number of organisms which can infect the host, for example, S. aureus (particularly, methicillin-resistant S. aureus, i.e., MRSA), E. coli, S. epidermidis (particularly methicillin-resistant S. epidermidis, i.e., MRSE), Pseudomonas aeruginosa, Enterococcus faecalis (including vancomycin-resistant Enterococcus faecalis, i.e., VREF) and Bacteroides sp. These and other aerobic and anaerobic pathogens can cause severe morbidity and death amongst large patient populations.

Implanted medical devices, such as heart valves and artificial veins and joints, are especially vulnerable to microbial biofilm formation and disease. Closed implants are more frequently associated with life-threatening situations, with S. epidermidis and S. aureus being the major pathogens. In S. aureus, exotoxins, siderophores and other substances make the organism able to infect the host. Urinary tract, vaginal and intestinal infections caused by E. coli can be serious, chronic or fatal. Each year, an estimated 150 million cases of urinary tract infection (UTI) resulting in significant patient morbidity and billions of dollars in health care expenditures. Although most UTIs possess a bacterial etiology, ˜75-80% of uncomplicated infections involve strains of E. coli. These strains have been termed as “uropathogenic” E. coli (UPEC) and demonstrated to produce multiple factors associated with the infectious process. Intestinal pathogenic E. coli can produce various toxins such as Shiga-like toxin that is lethal to some people. These virulence factors (VFs) include proteins involved in host cell attachment and invasion (e.g., fimbriae and adhesins), cytotoxicity (e.g., haemolysins and toxins), iron-acquisition (e.g., siderophores) and evasion or disruption of host-cell defences (e.g., capsule). Genes encoding these factors have been shown to be linked to plasmids and the distinct chromosomal regions that are termed pathogenicity islands.

Lactobacilli have long been known to play an important role in protecting the host from infections, such as urogenital infections. Studies have shown that these bacteria produce numerous substances such as organic acids, hydrogen peroxide, bacteriocins and biosurfactants that kill pathogens or inhibit their adherence to surfaces. The mode of action is through pH changes, steric hindrance, receptor blockage and direct killing. However, there is little known regarding the effects of Lactobacilli on the virulence of pathogens, such as urogenital pathogens.

### SUMMARY OF THE INVENTION

The present invention provides methods and compositions for altering the virulence or infectivity of pathogens in a mammal by administering to the mammal a therapeutically effective amount of at least one non-pathogenic microorganism in the form of viable cells able to signal alteration in the virulence or infectivity of the pathogens, or by administering at least one signal molecule produced by the non-pathogenic microorganism in a suitable form which confers the same alteration effect.

One aspect of the present invention is directed to the signal molecules that can alter the virulence or infectivity of pathogens in a mammal. Such molecules can be proteins or peptides that are produced by Lactobacillus or other organisms including, but not limited to, Bifidobacterium.

In the practice of the methods in the present invention, the pathogens can be from a group comprising S. aureus, Enterococcus, Streptococcus, Staphylococcus, Clostridium, Shigella, Salmonella, E. coli, Prevotella, Gardnerella, Klebsiella, Pseudomonas, Campylobacter, Candida, Proteus, Burkholderia. Mycobacterium, Helicobacter, Bacteroides, Vibrio, Listeria, Yersinia, Chlamydia, Meningococcus, Neisseria.

In a preferred aspect of the present invention, the Lactobacillus can be selected from the group consisting of L. rhamnosus, L. casei, L. acidophilus, L. fermentum, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, L. iners and L. bifidum. Preferably, the Lactobacillus is L. fermentum RC-14 or L. reuteri RC-14, Lactobacillus B54, L. jensenii PC1 or L. rhamnosus GR-1.

In another aspect, the methods of the present invention are further directed to the treatment or prevention of infections.

The present invention also provides a method for delivering signal molecules to medical devices or to sites surrounding medical devices in order to reduce the risk of infections associated with the medical devices.

In a preferred form of the present invention, the medical devices include, but not limited to, catheters, lines, stents, tubes, bags, valves, implants, instruments, and other materials which contain substances that could infect the host.

The present invention also provides a pharmaceutical composition suitable for treating and preventing infections in mammals, which comprises a therapeutically effective amount of at least one signal molecule in an acceptable carrier. The carrier can be in forms of a pharmaceutical carrier or natural foods. Preferably, the composition comprises proteins/peptides isolated from the group consisting of L. rhamnosus, L. casei, L. acidophilus, L. fermentum, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, L. iners, and L. bifidum. More preferably, the Lactobacillus is L. fermentum RC-14 or L. reuteri RC-14, Lactobacillus B54, L. jensenii PC1 or L. rhamnosus GR-1.

In yet another aspect of the present invention, a method is provided for reducing the symptoms and signs of infection caused by pathogens. In a preferred form of the present invention, such symptoms and signs include, but not limited to, sepsis, pain, bacteremia inflammatory bowel disease and general morbidity. The method for reducing the risk of death caused by pathogens is also provided by the present invention.

### BRIEF DESCRIPTIONS OF DRAWINGS

FIG. 1 depicts the co-culture experiment setup and procedures.

FIG. 2-FIG. 7 depict the 2D protein gel images from co-culture experiments of E. coli and Lactobacillus.

FIG. 8 depicts the 2D protein gel images from co-culture experiments of S. aureus Newman/Lactobacillus.

FIG. 9 depicts gene and encoded protein analysis of Exotoxin.

FIG. 10 depicts the detection of exotoxin gene on/off by a gfp-lux reporter.

FIG. 11 depicts the average OD/Lumi values which illustrate the on/off of exotoxin gene.

FIG. 12 depicts SET15 expression was down regulated by RC14 by products.

FIG. 13 and FIG. 14 depict that RC-14 by products suppress the promoter of SET15.

FIG. 15 and FIG. 16 depict that RC-14 by products suppress the P3 promoter.

FIG. 17 depicts that decrease of SET15 expression is independent of agr pathway.

FIG. 18 depicts the result of co-culture experiments of L. jensenii PC1 and E. coli. Total luminescence versus growth (OD 575 nm) for Escherichia coli C1212 reporter clones harboring constructs containing the virulence factor promoters for FimA, OmpA, OmpX and PapA upstream of the lux (luciferase) operon. Cultures contained 25% 4× modified MRS media (mMRS) and 75% 1× mMRS, 1× mMRS salts or 48 hour Lactobacillus SCS. Cultures were inoculated with 1×105 cells of the respective reporter clone and grown at 37 C for 24 hours.

FIG. 19 depicts 2-DE analysis of S. aureus Newman cell surface-associated proteins when co-cultured with media (FIG. 23A), L. reuteri RC-14 (FIG. 23B) or L. rhamnosus GR-1 (FIG. 23C) as described in experimental procedures. Spots were analyzed using Phoretix 2D software, and only spots with a fold of change greater than 2 were further analyzed. Proteins labeled in FIG. 23A and FIG. 23B, showed decreased and increased expression, respectively, relative to growth with RC-14. Note that protein 1 showed a dramatic decrease in expression in response to growth with RC-14, but not GR-1. This protein has a pl of ˜5.9 and a molecular weight of ˜25 kDa and was determined to be homologous to a known staphylococcal superantigen-like protein, SSL11, by mass spectrometry analysis. Molecular mass markers are indicated on the right and the pI gradient is shown on the bottom.

FIG. 20 depicts chematic representation of the ssl11 locus in S. aureus and characterization of SSL11. In FIG. 24A, arrows are representative of individual coding regions from S. aureus COL (Gill et al., 2005). The ssl11 gene is preceded by a 382-bp region lacking predicted ORFs, which is directly downstream of SA0447, a gene whose product is predicted to belong to a restriction endonuclease complex. SA0479 is predicted to encode a lipoprotein of unknown function. The nucleotide sequence of the intragenic region between SA0477 and ssl11 (nucleotides 479807-480191 in COL) is shown with the region cloned into pJLED1 shown in uppercase letters, and the start of SSL11 indicated with the corresponding amino acid sequence. FIG. 24B depicts ClustalW alignment (Thompson et al., 1994) of SSL11 alleles. Identical alleles are shown together and asterisks indicate identical residues. The first 30 amino acids are predicted to encode a classical signal peptide which is identical for all alleles.

FIG. 21 depicts repression of the ssl11 promoter by RC-14. FIG. 25A depicts relative light units (RLUs) detected from S. aureus Newman harboring either pJLED1, or pSB2034, grown either in control media (λ) or in L. reuteri RC-14 supernatant (◯). Results shown are from a representative experiment in which each condition was performed in triplicate. FIG. 25B depicts that maximum luminescence detected per maximum CFU detected from S. aureus Newman harboring either pJLED1 (a) or pSB2034 (b). OD₆₀₀ and RLUs were taken every hour over a period of 48 h. S. aureus harboring either gene reporter construct was grown in BHI media (BHI), L. reuteri RC-14 supernatant (SUP), in decreasing concentrations of L. reuteri RC-14 supernatant (25% SUP, 15% SUP, and 5% SUP), in pH adjusted L. reuteri RC-14 supernatant (pH SUP), catalase treated L. reuteri RC-14 supernatant (CAT. SUP), in BHI containing a small amount of concentrated L. reuteri RC-14 supernatant (CON. SUP) or in L. rhamnosus GR-1 supernatant (GR1) in a 96-well plate. The suppression of SSL11 promoter activation by L. reuteri RC-14 supernatant was also insensitive to protease treatment, thermal treatment, and a lactic acid effect. Error bars represent the standard error from 3 separate experiments done in triplicate. *P<0.05 vs. BHI. FIG. 25C depicts RLUs detected from S. aureus Newman harboring pJLED1 grown over a 48 hour time period. S. aureus was cultured either in BHI medium (λ), L. reuteri RC-14 supernatant (◯), or BHI medium supplemented with concentrated L. reuteri RC-14 supernatant at 26 h (σ). The vertical arrow denotes the time period at which concentrated supernatant was added to the BHI medium.

FIG. 22 depicts identification of monocytes and dendritic cells (DC) by flow cytometry. A representative example of the identification of monocytes based on the expression of CD33 (FIG. 26A) and CD14 antigen (FIG. 26B). FIG. 26C depicts identification of DC as an HLA-DR+ lineage negative (CD3−, CD56−, CD14−, CD19−) population. FIG. 26D shows that after acquiring a higher number of cells within the HLA-DR+lineage negative live gate, three different dendritic cell subsets were identified on the basis of CD33 expression: myeloid CD33_{high}, CD33_{intermed} and plasmocytoid CD33_{-/low}.

FIG. 23 depicts suppressive effect of cell-free extracts (CFE) of Lactobacillus reuteri RC-14 and Lactobacillus rhamnosus GR-1 on the in vitro proliferative responses of peripheral blood mononuclear cells (PBMC). PBMC obtained from 5 healthy controls were cultured with (+) or without (−) PMA, ionomycin and CFE. Results are expressed as mean optical density (OD) at 575 nm+SD, with higher OD corresponding to higher proliferation rate.

FIG. 24 depicts comparison of fold changes in the numbers of regulatory T-cells (Treg, CD4+CD25_{high}), activated T-cells (CD4+CD25+) and TNF-α and IL-12 producing monocytes (MC) and dendritic cells (DC) in IBD patients following treatment with probiotic-yogurt or unsupplemented yogurt. Individuals are indicated by connective lines. * Change following treatment with probiotic-yogurt significantly different from change following treatment with unsupplementd yogurt (p<0.05).

### DETAILED DESCRIPTION OF THE INVENTION

The present invention is directed to methods and compositions for altering the virulence or infectivity of pathogens in a mammal by administering to the mammal a therapeutically effective amount of at least one non-pathogenic microorganism in the form of viable cells that are able to alter the virulence or infectivity of the pathogens, or by administering at least one signal molecule produced by the non-pathogenic microorganism in a suitable form which confers the same alteration effect. The present invention also encompass altering the virulence or infectivity of pathogens in a mammal infected by the pathogens by administering to the mammal a therapeutically effective amount of non-pathogenic microorganisms in the form of viable cells that are able to alter the virulence or infectivity of the pathogens, or by administering signal molecules or by products produced by the non-pathogenic microorganisms in a suitable form, such as supernatant of the non-pathogenic microorganism culture, which confers the same alteration effect.

By “by product” or “signal(ing) molecule” is meant a molecule produced by a non-pathogenic microorganism, e.g., Lactobacilli or Bifidobacterium, which alters virulence or infectivity of a pathogen, e.g., pathogenic microorganism. The by product or signal molecule of the present invention can be a protein or peptide molecule, which is also termed as a “signal(ing) protein” or “signal(ing) peptide.” Specifically, the by product or signal molecule of the present invention can down regulate virulence properties, e.g., expression of virulent factors, of a pathogen. Signal molecules or by products, such as proteins, can be isolated by conventional methods that are well established in the art. The characteristics of a signal molecule can be tested by in vitro experiments, such as co-culturing experiments described in the present invention, as well as by in vivo experiments. For example, proteins isolated from the supernatant of a non-mircoorganism culture can be applied to a locus of a mammal, such as urinary, that is infected by a pathogen. A signal protein that down regulates the virulence of the pathogen will reduce the infection.

By “pathogen” is meant a microbe or other organism that causes disease. Pathogens of the present invention can be pathogenic microorganisms or microbial biofilms, including, but not limited to, bacteria, fungi, protozoa, viruses and other forms, or organisms which behave like pathogens under certain situations, such as host defense compromise.

By “virulence factor” or “VF” is meant a factor, molecule, structure or mechanism that causes a pathogen having capacity to cause disease, such as adhesins which colonize tissues and surfaces, toxins, slime and other capsular substances, antibiotic-resistance genes, immune modifiers and substances which help escape immune responses. Virulence factors (VFs) of referred to in the present invention include, but not limited to, proteins involved in host cell attachment and invasion (e.g., fimbriae and adhesins), cytotoxicity (e.g., haemolysins and toxins), iron-acquisition (e.g., siderophores) and evasion or disruption of host-cell defences (e.g., capsule).

By “pathogenicity islands” is meant plasmids and the distinct chromosomal regions that comprise genes encoding VFs.

By “treat,” “treatment” or “treating” is meant to ameliorate, suppress, mitigate or eliminate the clinical symptoms after the onset (i.e., clinical manifestation) of diseases caused by pathogens, such as urinary tract infection. An effective or successful treatment provides a clinically observable improvement.

By “prevent,” “preventing” or “prevention” is meant that the risk of having an abnormality, disorder or disease, such as urinary tract infection, can be predicted or determined in sufficient time so as to keep the disorder or abnormality from occurring or significantly reduce the risk of having the abnormality, disorder or disease.

One embodiment of the present invention is directed to the signal molecules, e.g., proteins or peptides, that are produced by Lactobacillus or other organisms including, but not limited to, Bifidobacterium. In a further embodiment of the present invention, these signal molecules can alter virulence or infectivity of pathogens, e.g., by down regulating gene expression of uropathogenic bacterial virulence factors.

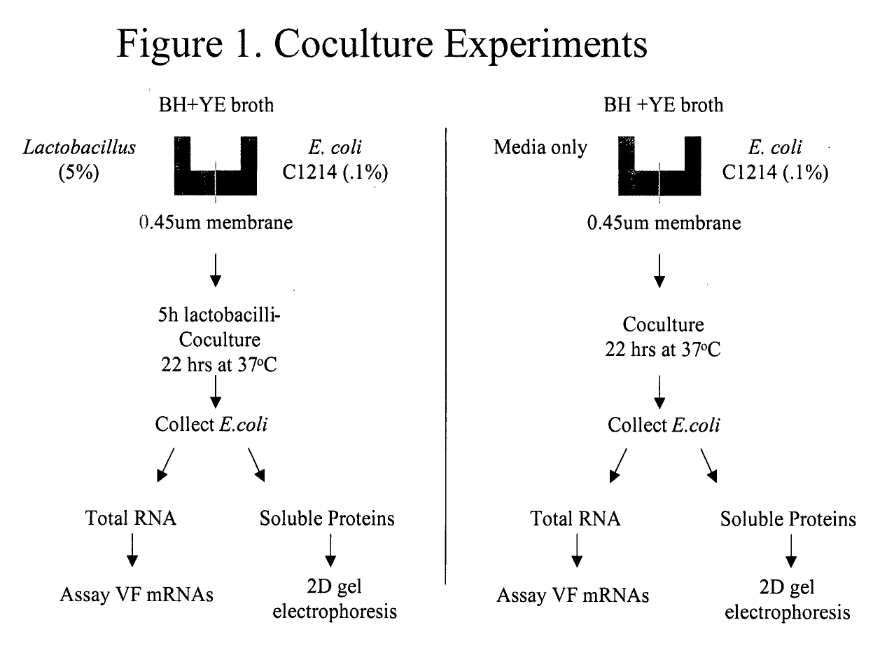

According to the present invention, it has been found that the presence of Lactobacilli cells or their by-products in the vicinity of an uropathogenic bacterium, for example, E. coli, can down regulate virulence production by the uropathogenic bacterium, e.g., fimbria production by E. coli. As illustrated below in Examples, the live Lactobacilli and E. coli cultures are separated by a filter (see, e.g., FIG. 1 and Example 3), which only allows by-products to pass through and induce the down regulating reaction. Without intending to be limited to a particular theory, it is believed that Lactobacilli by-products are signal molecules that can alter the infectivity of pathogens, such as pathogenic microorganisms, in a mammal by down regulating virulence production by the uropathogenic bacteria, preferably by altering or down regulating gene expression of uropathogenic bacterial virulence factors. For example, the present invention demonstrates that the signal molecules alter the expression of exotoxin genes located in the pathogenicity island of the Staphylococcus aureus genome. Example 10 further demonstrates that L. reuteri RC-14 is able to decrease the virulence potential of S. aureus via cell-cell signalling molecules. Without intending to be limited by any particular theory, it is believed that the the signal molecules produced by Lactobacilli or other non-pathogenic microorganisms are protein or peptide molecules. In a preferred embodiment, the signal molecules of the present invention are heat labile and are not acid or hydrogen peroxide.

In one particular embodiment, the present invention encompasses the infections caused by biofilms.

While Lactobacilli strains have long been known to produce acids, hydrogen peroxide, bacteriocins and other substances that can kill other organisms, it has been discovered for the first time by the present invention that the Lactobacilli can produce signal molecules that can down regulate virulence factors produced by pathogens.

Without intending to be limited to a particular theory, it is believed that the pathogens need not be killed for the infection to be curtailed, treated or prevented. For example, as illustrated in Examples 2-3, the presence of by-products from L. fermentum RC-14 strain does not inhibit the growth of the pathogens (see Table 3) but the infection of the pathogens is curtailed or altered. This mechanism is contrary to antibiotics, antimicrobials, bacteriocins and other previously identified mechanisms designed to kill or attack the growth of the organisms. For example, bacteriocins range from a simple protein to a high molecular weight complex. The genetic determinants of bacteriocins are mostly located on plasmids. The action of bacteriocins is species specific whereby bacteriocins exert lethal activity through adsorption to specific receptors located on the external surface of sensitive bacteria, followed by metabolic, biological and morphological changes resulting in the killing of such bacteria. On the contrary, in the present invention, growth of the pathogens per se need not be necessarily altered at all. Rather, the ability of the pathogens to damage the host is curtailed.

According to the present invention, alterations or down regulation of pathogens' virulence can affect Gram positive and Gram negative pathogens as well as candida and yeast cells. Accordingly, in another embodiment of the present invention, the pathogens of the present invention can be Gram positive or Gram negative bacteria.

In a particular embodiment of the present invention, the pathogens can be selected from a group comprising S. aureus, Enterococcus, Streptococcus, Staphylococcus, Clostridium, Shigella, Salmonella, E. coli, Prevotella, Gardnerella, Klebsiella, Pseudomonas, Campylobacter, Candida, Proteus, Burkholderia. Mycobacterium, Helicobacter, Bacteroides, Vibrio, Listeria, Yersinia, Chlamydia, Meningococcus, or Neisseria.

In another particular embodiment of the present invention, the Lactobacillus can be selected from the group consisting of L. rhamnosus, L. casei, L. acidophilus, L. fermentum, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, L. iners and L. bifidum. Preferably, the Lactobacillus is L. fermentum RC-14 or L. reuteri RC-14, Lactobacillus B54, L. jensenii PC 1 or L. rhamnosus GR-1.

The methods of the present invention are further directed to the treatment or prevention of infections.

By “therapeutically effective amount” as used in the present invention is meant an amount of Lactobacillus or by-product thereof, high enough to significantly positively modify the condition to be treated but low enough to avoid serious side effects (at a reasonable benefit/risk ratio), within the scope of sound medical judgment. An effective amount of Lactobacillus or by-product thereof can vary with the particular goal to be achieved, the age and physical condition of the patient being treated, the race, the severity of the underlying disease, the duration of treatment, the nature of concurrent therapy and the specific Lactobacillus or by-product thereof employed. The effective amount of Lactobacillus or by-product thereof will thus be the minimum amount which will provide the desired anti-infection effect. For example, the presence of about 1×¹⁰⁹ bacteria, as viable whole cells, in about 0.05 ml solution of phosphate buffered saline solution, or in about 0.05 ml of suspension of microbial nutrients or prebiotics, or the dry weight equivalent of cell wall fragments, is effective when administered in quantities of about 0.05 ml to about 20 ml. However, the presence of about 0.05 ml to about 20 ml of Lactobacillus by-product solution in MRS broth produced from about 1×10⁹ bacteria is also effective.

A decided practical advantage is that the Lactobacilli or by-products thereof can be administered in a convenient manner such as by the intravenous (where non-viable), suppository (vaginal or rectal) routes. Depending on the route of administration, the active ingredients which comprise the Lactobacilli may be required to be coated in a material to protect the organisms from the action of enzymes, acids and other natural conditions which may inactivate said organisms. However, in the case of by-product administration, carriers rather than coatings may be required.

In one embodiment of the present invention, the by-products of Lactobacilli are administered topically or by coating or partially coating the portion of the biosurface or biomaterial that is inserted or placed into the desired locus, e.g., the urinary or vaginal epithelia. Any common topical formulation such as a solution, suspension, gel, cream, ointment, or salve and the like may be used. Preparation of such topical formulations is well described in the art of pharmaceutical formulations as exemplified, for example, in Remington's Pharmaceutical Science, Ed. 17, Mack Publishing Company, Easton, Pa. (1988).

A particular embodiment of the present invention contemplates administering the by-products of Lactobacilli via diapers, sanitary pads or feminine pads, feminine tampons, facial creams, wound dressings and oral products.

The present invention also provides a method to deliver or apply Lactobacilli and/or signal molecules thereof to medical devices or to the sites surrounding the medical devices in order to reduce the risk of infections associated with the devices.

In a preferred form of the present invention, the devices include catheters, lines, stents, tubes, bags, valves, implants, instruments, and other materials which contain substances that could infect the host.

The present invention also provides a pharmaceutical composition suitable for preventing infections in mammals. The pharmaceutical composition of the present invention comprises a therapeutically effective amount of at least one of the signal molecule, e.g., a protein or peptide molecule, in an acceptable carrier, which can be a pharmaceutical carrier or natural foods. Preferably, the composition comprises proteins or peptides isolated from the group consisting of L. rhamnosus, L. casei, L. acidophilus, L. fermentum, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, L. iners, and L. bifidum. More preferably, the Lactobacillus is L. fermentum RC-14 or L. reuteri RC-14, Lactobacillus B54, L. jensenii PC1 or L. rhamnosus GR-1.

In yet another embodiment of the present invention, a method is provided for reducing the symptoms and signs of an infection in a mammal caused by pathogens by administering to the mammal a therapeutically effective amount of non-pathogenic microorganisms and/or signal molecules produced by such non-pathogenic microorganisms. The infection-causing pathogens include, but not limited to, bacteria, viruses, protozoa. In a preferred embodiment, the non-pathogenic microorganism employed is Lactobacillus. In a more preferred embodiment, the Lactobacillus is L. fermentum RC-14 or L. reuteri RC-14, Lactobacillus B54, L. jensenii PC1 or L. rhamnosus GR-1. The symptoms and signs encompassed by the present invention include, but are not limited to, sepsis, pain, bacteremia, inflammatory bowel disease (see Example 11) and general morbidity. In particular embodiment, the present invention provides a method for reducing the risk of death caused by pathogens by administering a therapeutically effective amount of non-pathogenic microorganisms and/or signal molecules produced by such non-pathogenic microorganisms.

The examples below are offered by way of example, and are not intended to limit the scope of the present invention in any manner.

### EXAMPLE 1

Virulence factors (VFs) produced by bacteria are exemplified below, in which the expression of virulence factors in a number of UPEC strains was confirmed (see Table 1). Oligonucleotide primers specific for the genes encoding VFs, such as type 1, P, F1 C and S fimbriae, haemolysin A, aerobactin, afimbrial adhesin I (afa I), cytotoxic necrotizing factors I and II (cnfs I and II), KII and KIII capsular proteins and 16S rRNA (control), were generated and PCR was carried out to screen for the presence of these genes. One PCR product for each VF was sequence verified. To confirm the PCR results, sequenced amplicons were also DIG-labelled and used as probes to screen genomic DNA of all the E. coli strains by dot-blot hybridization. The expression of haemolysin A and type 1 pili were also determined by plating on Columbia agar and haemagglutination assays, respectively.

TABLE 1
Bacterial Strains Used in This Study

| | | | Previously |
|---|---|---|---|
| | | | Identified |
| Bacterial Species | Strain | Human Origin | Virulence Factors |
| Escherichia coli | Co1 | Faeces | None |
| Escherichia coli | Hu734 | Pyelonephritis | Type 1 Fimbriae, |
| | | | MRA |
| Escherichia coli | 67 | UTI | P fimbriae |
| Escherichia coli | 431 | UTI | None |
| Escherichia coli | 917 | UTI | Type 1 and P |
| | | | fimbriae |
| Escherichia coli | C1212 | UTI | Type 1 Fimbriae, |
| | | | MRA |
| Escherichia coli | C1214 | UTI | Type 1 Fimbriae |
| Lactobacillus rhamnosus | GR-1 | Vagina | None |
| Lactobacillus reuteri | RC-14 | Vagina | None |

UTI—Urinary tract infection.
MRA—Mannose-resistant adhesion, which refers to any adhesive molecule whose expression induces mannose-resistant hemagglutination in a bioassay.

Both PCR and DNA dot-blot hybridization analyses gave similar results for the detection of VF genes among the E. coli strains (Table 2). The control fecal isolate E. coli Co1 was found only to harbor type 1 fimbrial genes, the sole VF assayed that was common to all strains. Iisolates E. coli C1212 and E. coli C1214 harbor the greatest number of VF genes, encoding type 1, P and F1 C fimbriae, as well as aerobactin, haemolysin A and cnf I (E. coli C1214 only). Genes encoding afa I and S fimbriae were only detected in E. coli 431 and E. coli 67, respectively. None of the strains in the study were found to carry genes associated with cnf II or KIII capsular proteins. Expression of haemolysin A was also observed for E. coli C1212 and E. coli C1214 via culturing on Columbia agar plates, a finding consistent with the genetic screening results. Haemagglutination assays demonstrated type 1 fimbrial expression in all of the E. coli strains except E. coli 431, even though genetic screening determined the presence of type 1 genetic material.

TABLE 2
UPEC Virulence Factor Genetic Screening Results

| | E. coli Strains | | | | | | |
|---|---|---|---|---|---|---|---|
| Virulence Factors | Co1 | Hu734 | 67 | 431 | 917 | C1212 | C1214 |
| None (16S rRNA | + | + | + | + | + | + | + |
| control) | | | | | | | |
| Type 1 Fimbriae | + | + | + | + | + | + | + |
| P Fimbriae | − | + | − | − | + | + | + |
| Afimbrial | − | − | − | + | − | − | − |
| Adhesin I (AfaI) | | | | | | | |
| Aerobactin | − | + | − | − | + | + | + |
| Siderophore | | | | | | | |
| Cytotoxic | − | − | − | − | − | − | + |
| Necrotizing | | | | | | | |
| Factor | | | | | | | |
| I (cnfI) | | | | | | | |
| Cytotoxic | − | − | − | − | − | − | − |
| Necrotizing | | | | | | | |
| Factor | | | | | | | |
| II (cnfII) | | | | | | | |
| Hemolysin A | − | − | − | − | − | + | + |
| S Fimbriae | − | − | + | − | − | − | − |
| F1C Fimbriae | − | − | − | − | − | + | + |
| (focG) | | | | | | | |
| Capsule | − | + | + | − | + | − | − |
| (kpsMT II) | | | | | | | |
| Capsule | − | − | − | − | − | − | − |
| (kpsMT III) | | | | | | | |

+ Positive for virulence factor gene as determined via PCR and DNA dot-blot hybridization;
− Negative

### EXAMPLE 2

To initially assess the effects of Lactobacillus secreted by-products on UPEC growth, differential antagonism and well diffusion qualitative assays were employed. The Lactobacillus strains Lactobacillus rhamnosus GR-1 and L. reuteri RC-14 were grown as 1 cm wide lawns on BHIS agar for 48 hours. These lawns were then removed, perpendicular 1 cm lawns of each UPEC strain were plated across the original Lactobacillus streaks and plates were incubated 16-18 hours and the growth was assessed. Well diffusion assays involved testing spent culture supernatants (SCS) isolated from both Lactobacillus strains grown for 48 hours in BHIS broth on the growth of the UPEC strains. Lactobacillus SCS were pipetted into wells cut out of agar plates harboring surface lawns of the UPEC strains. The plates were incubated for 16-18 hours and the growth was assessed. Additionally, supernatants were either boiled for 10 minutes, neutralized to pH 7.0, treated with catalase or treated with proteinase K prior to use in the assay to determine whether the observed effects were due to a heat labile compound, low pH, hydrogen peroxide or a proteinaceous factor, respectively.

Both differential antagonism and well diffusion assays demonstrated moderate to complete growth inhibition of all E. coli strains when grown in the presence of L. rhamnosus GR-1 by-products and zero to slight inhibition when grown in the presence of those from L. reuteri RC-14 (Table 3). Treatment of the culture supernatants with heat, catalase or proteinase K did not alter the findings. However, neutralization of the supernatants resulted in their complete loss of inhibitory activity.

TABLE 3
Lactobacillus-Induced Growth Inhibition of UPEC Strains

| | Growth Inhibition | | Growth Inhibition | |
|---|---|---|---|---|
| | (Differential Antagonism) | | (Well Diffusion) | |
| | L. rhamnosus | L. reuteri | L. rhamnosus | |
| E. coli | GR-1 | RC-14 | GR-1 | L. reuteri |
| Strain | by-products | by-products | SCS | RC-14 SCS |
| Co1 | +++ | + | ++ | + |
| Hu734 | +++ | − | + | + |
| 67 | +++ | + | ++ | + |
| 431 | +++ | + | ++ | + |
| 917 | ++ | − | ++ | − |
| C1212 | ++ | − | ++ | − |
| C1214 | ++ | − | ++ | − |

Differential Antagonism:
− no inhibition of E. coli growth over Lactobacillus by-products;
+ slight inhibition (≦25% growth reduction);
++ moderate inhibition (25-75% growth reduction);
+++ strong inhibition (75-100% growth reduction).
Well Diffusion:
− no E. coli growth inhibition surrounding well;
+ ≦1.0 mm halo of inhibition;
++ 1.0-2.0 mm halo of inhibition;
+++ 2.0-3.0 mm wide halo of inhibition.
Notes:
MRS media was used as the control for both assays and produced no growth inhibitory effects on any of the E. coli strains. The results are the average of three experiments.
“SCS”: spent culture supernatent.

These findings demonstrate that the incidence of VF genes widely varies among UPEC isolates. Additionally, by-products isolated from Lactobacillus, e.g., rhamnosus GR-1, can inhibit the growth of UPECs, most likely due to changes in pH.

This inhibition is confirmatory in that previous studies have shown that Lactobacilli can inhibit the growth of pathogens (Reid et al. J Urol. 138:330-35, 1987). Nevertheless, the present invention discovers that other events take place between Lactobacilli and pathogens, i.e., those due to signaling.

### EXAMPLE 3

Using an experimental set up shown in FIG. 1, the ability of Lactobacillus-derived substances to affect the virulence of E. coli was demonstrated.

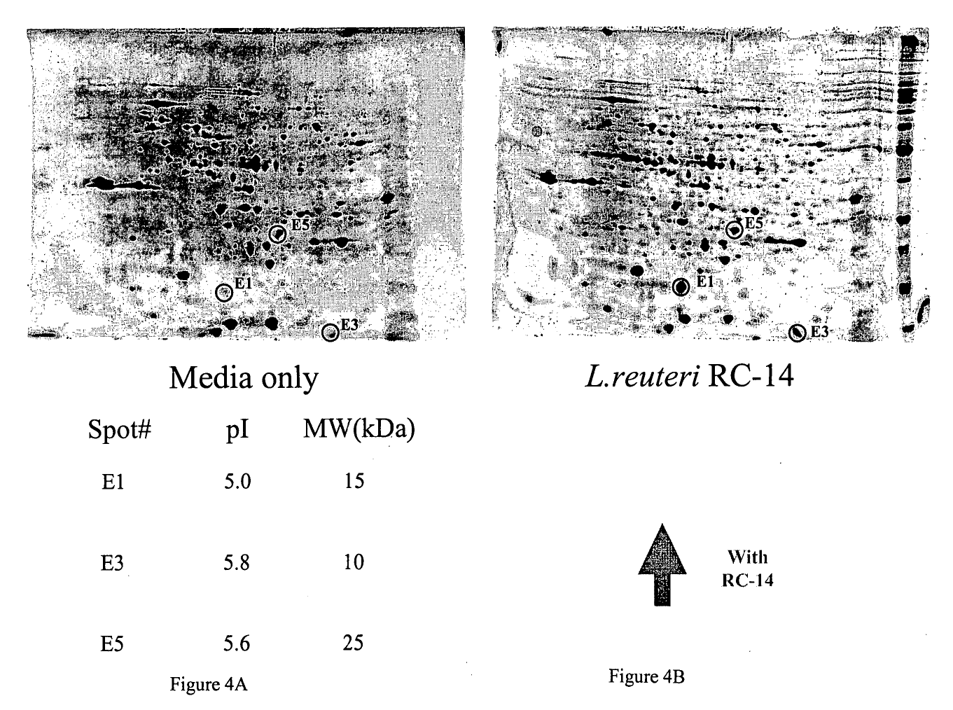

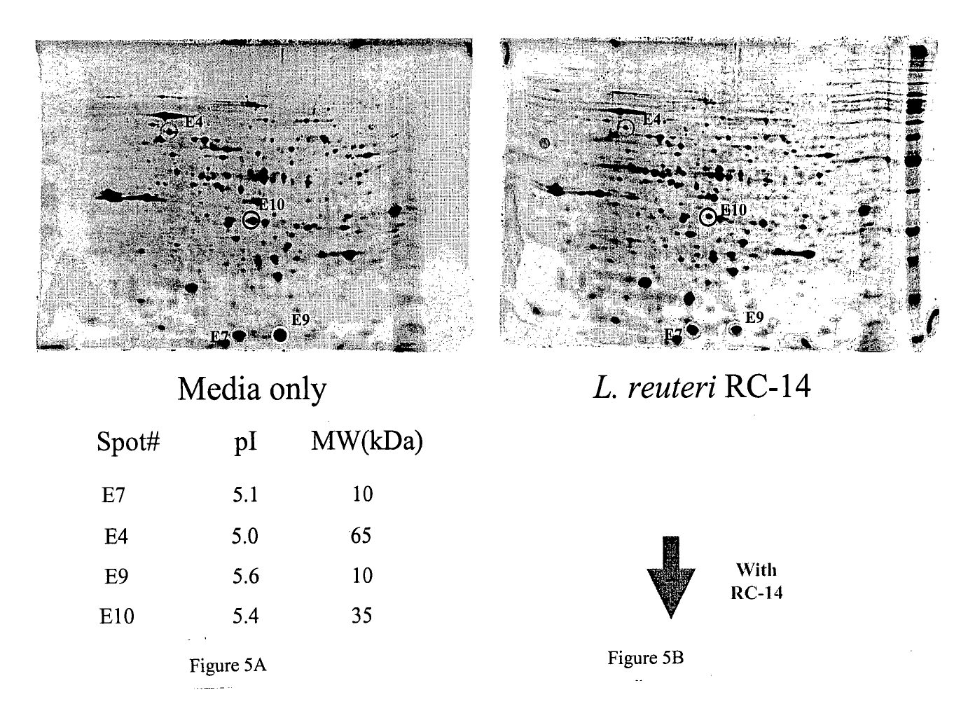

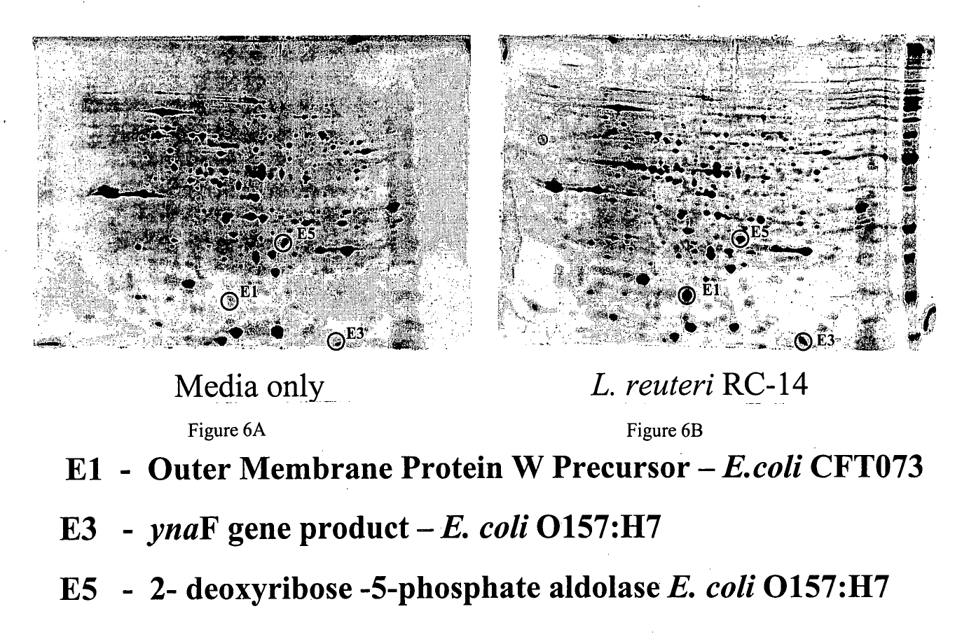

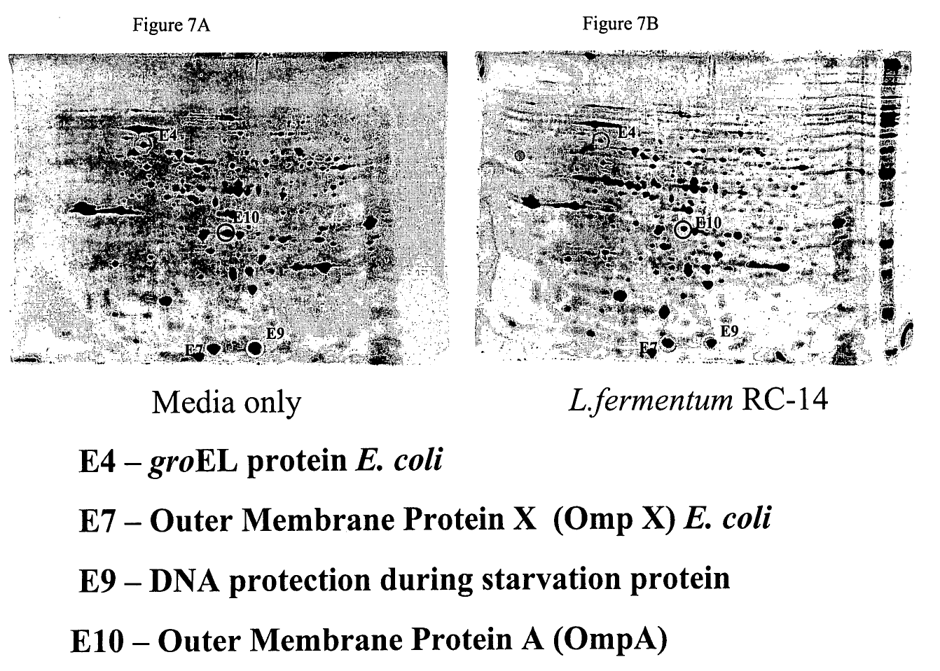

As shown in FIG. 1, the coculture of Lactobacillus and E. coli are separated by a 0.45 um membrane, which only allows molecules, not cells, to pass through. Thus, only by-products of the bacteria were able to induce the reaction or the bacterial “cross talk.” The first evidence of such bacterial “cross-talk” was obtained from a dot blot, which showed changes in S fimbrial expression caused by incubation with Lactobacillus GR-1. Similar changes were also observed from SQ-RT-PCR results, which showed that expression of VFs were altered, either up regulated or down regulated, when the E. coli strains were co-cultured with Lactobacillus GR-1 or RC-14. FIG. 4 to FIG. 9 illustrate the 2D protein gel results, which demonstrated that the expression of a uropathogenic E. coli VFs, such as fimbria, were altered when the E. coli were co-cultured with Lactobacillus.

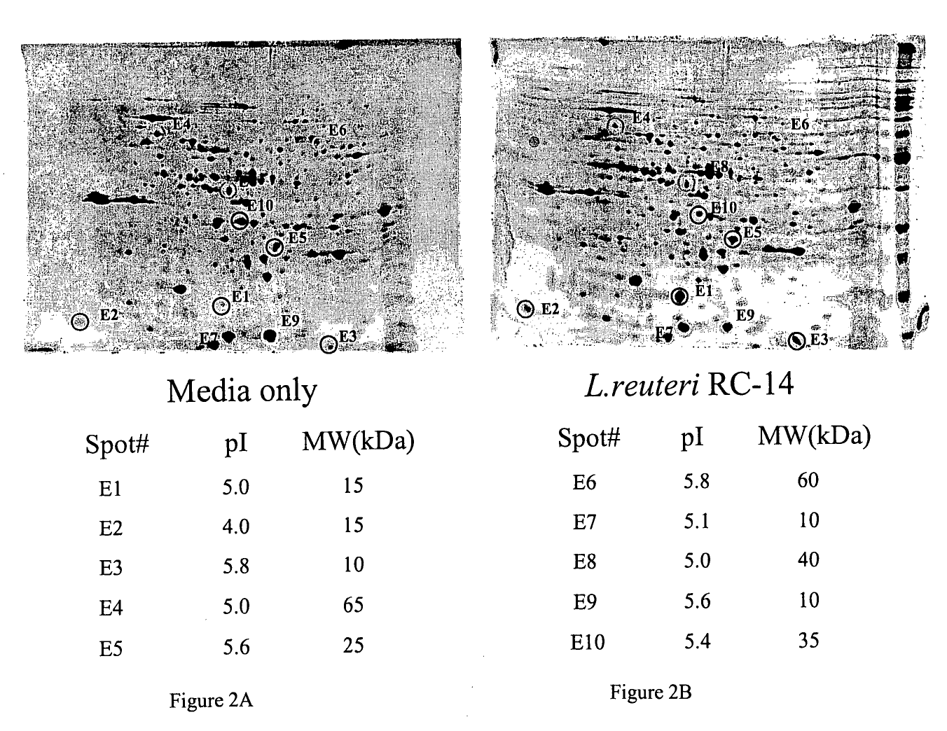

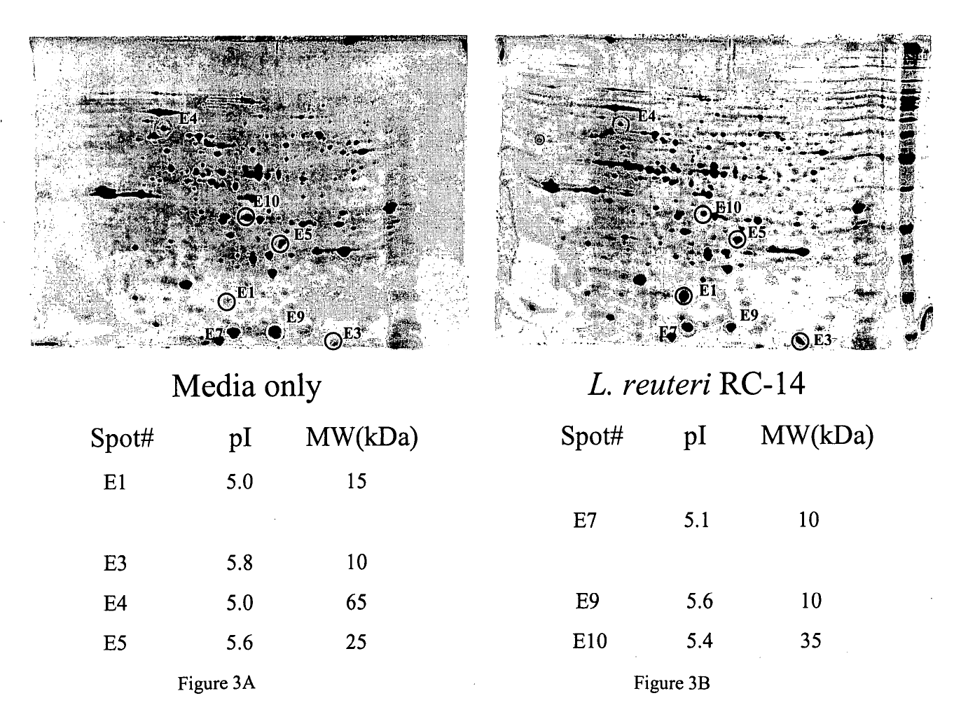

The spots E1-E10 identified in the 2D gels, as shown in FIGS. 2-7, are soluble proteins of the uropathogenic E. coli. Some of them are VF proteins. Characteristics of E1, E3-E5, E7 and E9-E10 are listed as follows:
E1 Outer Membrane Protein (Omp) W Precursor—E.coli CFT073
   23kDa minor protein of the E. coli outer membrane; -encoded by the yciD gene
   receptor for colicin S4;—plasmid encoded proteins synthesized by E. coli that kill sensitive strains—no OmpW, no S4 killing
E3 ynaF gene product—E. coli O157:H7
   18.4 kDa nucleotide binding protein—unknown function
   similarity to ATP-binding protein and universal stress protein
   may be induced in response to bacterial stress
E4 groEL protein-part of a family of ubiquitous proteins
   plays an essential role in ensuring proper three dimensional folding of proteins
   present in the cytoplasm of unstressed E. coli cells—expression increases during heat shock, nutrient deprivation, infection and inflammatory reaction to stabilize proteins
   implicated in bacterial disease pathogenesis
E5 2-deoxyribose-5-phosphate aldolase E. coli O157:H7
   27.7 kDa class I aldolase—forms a dimer
   catalyzes the generation of 2-deoxyribose-5-phosphate from acetaldehyde and D-glyceraldehyde 3-phosphate
   thought to function in deoxyribonucleoside catabolism
E7 Outer Membrane Protein X (Omp X) E. coli-integral outer membrane protein—16.3 kDa
   promotes bacterial adhesion and entry into mammalian cells
   also may play a role in complement resistance
   OmpX homologues found in Enterobacter, Klebsiella, Salmonella and Yersinia-overproduction downregulates OmpF and OmpC, resulting in b-lactam resistance
E9 protein for DNA protection during starvation.
E10 Outer Membrane Protein A (OmpA) E. coli CFT073-35 kDa major outer membrane protein—contributes to structural integrity of outer membrane
   important in protecting cells from environmental stresses
   colicin receptor and required in F-conjugation
   plays a key role in the invasion process of brain microvascular endothelial cells causing meningitis in neonates
   expression is reduced when OmpW overexpressed

In summary, the experiments here showed that the alteration of the expression of proteins, such as virulence factors, in E. coli is due to signaling molecules produced by Lactobacilli. For example, the experiments demonstrated that expression of OmpW (E1), ynaF (E3) stress protein and aldolase (E5) was up regulated when cocultured with RC-14. Expression of groEL (E4) stabilizer, DNA protection protein (E9), OmpX (E7) and OmpA (E10) was down regulated when co-cultured with RC-14.

### EXAMPLE 4

This exemplifies the use of bacterial compounds to affect virulence properties, again using a chamber that separates viable cells from each other. It demonstrated, for the first time, that Lactobacilli and/or its by products signal the down regulation of other virulence factors, e.g., in Staphylococcus aureus.

I. Bacterial Strains:

Staphylococcus aureus Newman

Staphylococcus aureus RN4220

Lactobacillus reuteri RC14

Lactobacillus rhamnosus GR1

Escherichia coli DH5a

II. Growth Conditions

(1) Monocultures of S. aureus or L. reuteri RC14 were grown in Brain Heart Infusion (BHI) broth, in anaerobic conditions (max volumes in the tubes, static).

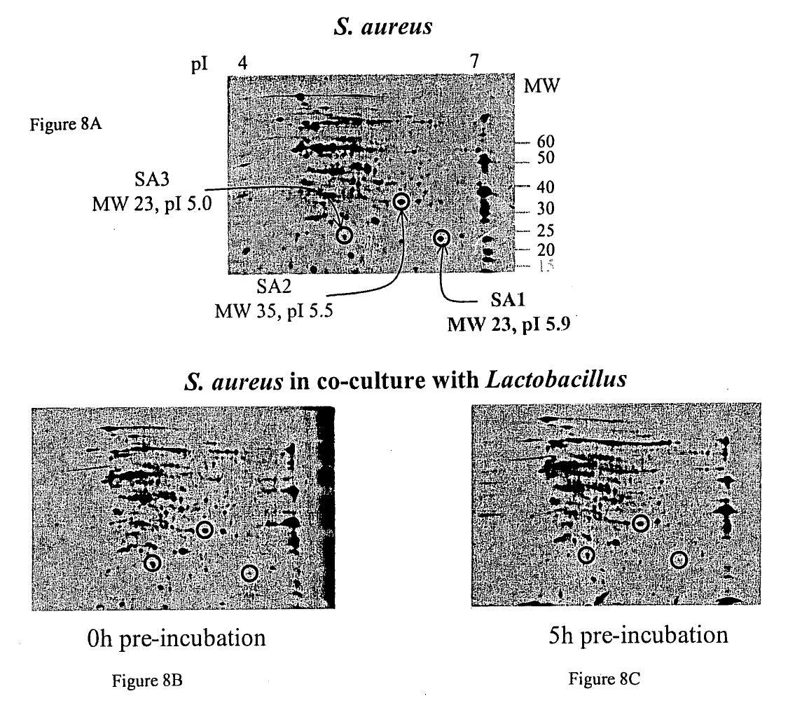

(2) Coculture experiment were carried out using two-compartment devises (provided by INRA, France). See FIG. 8. The two sterile compartments (30 ml each) were separated by a 0.45 um mixed cellulose esters membrane (Millipore). Cocultures were grown in BHI, at 37 C with a very slow shaking.

Different Conditions Were Tested:

(1) L. reuteri RC-14 Was Grown for 0, 1, 2 or 5 h Before Inoculum of S. aureus
Different inoculum sizes: 1, 2 or 5% inoculum were used
Coculture conditions were used with S. aureus inoculum: 1%
Coculture for 5 h (late log of S. aureus growth) or overnight (late exponential)

Only molecules can cross the membrane, not bacterial cells. The culture was checked with selective media (Mannitol for S. aureus and Rogosa or MRS for L. reuteri RC14, in both aerobically and anaerobically conditions). The culture was measured at OD600 nm at the end of the cocultures. When the best conditions were determined, the same experiment was repeated with L. rhamnosus GR1.

(2) Preparation of the Supernatant of L. reuteri RC14

L. reuteri RC14 was grown overnight in BHI to reach an OD600 of 0.4-0.6. This was reached by using C14 culture cultivated several times on BHI, before this final cultivation. The bacteria were then harvested (10 min, 5000 g, 4 C). The supernatant was filter sterilized using 0.2 um filters, and checked for sterility by plating an aliquot on MRS plates (anaerobic conditions). An aliquot was also used to check the pH of the supernatant.

The same supernatant was used to prepare heat inactivated supernatant. The supernatant was boiled for 10 min, and the volume brought back to the original volume by adding water (to compensate the loss by evaporation). This preparation was then filter sterilized and check for sterility. Also ‘neutralized’ supernatant was tested. This fraction was simply obtained by bringing the pH of the supernatant to the pH of BHI, using 5 N NaOH. This preparation was then filter sterilized and check for sterility.

(3) Growth of S.aureus in L. reuteri RC4 Supernatant

S. aureus was grown either in BHI, in L. reuteri RC14 supernatant undiluted or diluted ½in BHI.

The growth was monitored for at least 24 hours.

III. Preparation of the Proteins Extracts

I. Extraction of Cell Surface Associated Proteins (‘Cell Wall’ of Gram Positive Bacteria)

S. aureus cells were harvested (10 min, 5000 g, 4 C) and washed twice in cold 50 mM Tris pH 7.4. The supernatant was stored at −20 C. The cell surface associated proteins were extracted with sarkosyl. The pellet of cells (30 ml culture) was resuspended in 300 ul of Sarkosyl buffer (freshly prepared) (50 mM Tris pH 7.5, 150 mM NaCl, 1 mM MgCl₂, 2% N-lauroyl sarcosine). The suspension was incubated for 20 min on ice, and then centrifuged for 10 min at 14000 rpm. The pellet containing the protoplast was resuspended in 300 ul of 50 mM Tris pH 7.4 and stored at −20 C. The supernatant containing the cell surface associated (SA) proteins was used for the two-dimensional gel electrophoresis.

II. Removal of Contaminants

To remove salts, sugars, the proteins were precipated by an acetone-based procedure, using the Perfect Focus kit (GenoTech). Briefly 300 ul of sarkosyl preparation were precipitated and the pellet was resuspended in 100 ul of IEF buffer. To resuspend the proteins easily, incubate the pellet with the buffer at 37 C (not more than 50 C, urea+protein+heat=carbamylation of the proteins) for 30 min, vortexing some time to time. To remove any particules, centrifuge for 1 h at 15 C (the urea would precipitate at 4 C), and take the supernatant (=the fraction for the 2D gels). IEF buffer: 9 M urea, 4% CHAPS, 0.4% Ampholytes, 0.3% DTT Buffer is filtered on 0.2 um filter. It can be stored at −20 C (DTT being added just prior use).

III. Protein Assay (Modified Bradford)

Volume of standard or sample used for the assay: 15 ul
Standard: BSA 1 mg/ml (=1 ug/ul), made in IEF buffer
   From 0 to 15 ug
Samples: duplicate 5 ul or 10 ul (complete to 15 ul with IEF buffer)
Add 1 ml of Bradford reagent freshly prepared:
   5 ml undiluted reagent
   20 ml water
   20 ul HCL 12 N
Incubate for 15 min.
Read OD at 595 nm.
   III. Two-dimensional Electrophoresis
   IV. IPG Strip Rehydration

Strips were purchased from Biorad (or Pharmacia). They were 7 cm long and had a pH range of 4 to 7.

The samples (5-10 ug for analytical gels and up to 200 ug for preparative gels) were applied directly during the overnight rehydration step.

The rehydration was done in the focusing tray. First, the sample (125 ul total in IEF buffer, containing 1 ul of BromoPhenolBlue) was loaded in the well, then the strip (acidic end at the +) was put on the top of the liquid, and then everything was covered with mineral oil.

Rehydration conditions: active (50 V), overnight (16 h).

After the rehydration, wicks (soaked in water, and then excess of water is removed on Kim Wipes) were placed between the ends of the strips and the electrodes.

V First Dimension: Electrofocusing

These are the steps of the focusing:

S1: 200V for 100VH (rapid)

S2: 500V for 250VH (rapid)

S3: 1000V for 500VH (rapid)

S4: 8000V for 8000VH (rapid)

S5: 500V hold

The current is set up to a maximum of 50 uA/strip. For the last step, the voltage will may be not reach the 8000V, but it should be at least 5000V.

VI. Equilibration of the Strips

Equilibration buffer (must be filtered and then aliquoted and stored at −20 C)

6M urea

2% SDS

50 mM Tris HCl pH 8.8

30% glyceraol

Some BPB

Use a tray or tubes

First step of the equilibration:
   15 to 30 min in Equilibration buffer containing 1% DTT
Second step of the equilibration:
   15 to 30 min in Equilibration buffer containing 2.5% Iodoacetamide
      VII. Second Dimension: SDS-PAGE

Prepare SDS-PAGE as for a one-dimension electrophoresis: 10% Tris-Glycine
You need to use at least 1 mm thick gels
   All the solutions used are filtered to remove any particules.
   The stacking can be very small, use a special 2D comb.
   When stacking gel is polymerised, wash the top with Running Buffer
   Running buffer 10×:
   30.3 g Tris, 144 g glycine, 20 g SDS, Qsp 1000 ml
   Put the strips at the top of the stacking gel.
   Avoid any bubbles between the strip and the top of the gel.
   You can soak the strip in Running Buffer to help to position the strip.
   Embed the strip in agarose 0.8% in running buffer (melted agarose kept at 50-55 C).
Running conditions:
   20-30 min at 80V
   1 h 15 min at 120V
Staining: Sypro Ruby (Biorad) (sensitivity: same than Silver staining)
   All the steps are at room temperature, shaking
Fixation: 30 min in 10% MeOH, 7% Acetic acid
   Staining: overnight in undiluted Ruby (Ruby can be reused once)
   Destaining: at least 3 h (up to 24 h) in 10% MeOH, 7% Acetic acid.
   Store the gels in 5% acetic acid at 4 C

When the gels are used for mass spectrometry analysis, the quantity of samples has to be much higher (at least 50 ug) and the spot to analyse must be stained with Coomassie.
Fixation: 30 min in 50% methanol, 10% acetic acid
Staining: 30 min to 1 h in 50% methanol, 10% acetic acid with 0.1% Brilliant
Blue R250 (filtered before use)
Destaining in 50% methanol, 10% acetic acid until no background (about 2-3 h) Store the gels in 5% acetic acid at 4 C.
   VIII. Gel Image Analysis

The gel images were captured using an AlphaInnotech camera. The images of the gels are illustrated in FIG. 8. Each culture condition was repeated at least three times. The gel images were then analysed using the 2D Phoretix system. Briefly, average gels were created for each conditions. The spots were detected on the different average gels and the volume of each spot were calculated and divided by the total volume of all the spots on the gel of interest.

V. Identification of the Proteins

Trypsin Digestion

The protocol used for the trypsin digestion was the one provided by the UWO Biological Mass Spectrometry and can be found at the following web site: http://www.biochem.uwo.ca/wits/bmsl/bmslhome.html

Another Description of the Experimental Protocol:

Staphylococcus aureus strains Newman was grown on Brain Heart Infusion (BHI) broth in anaerobic conditions at 37 C. For the genetic manipulation of S. aureus Newman, a restriction-deficient derivative strain (strain RN4220) was used, both strains being grown on TSB. Lactobacillus reuteri RC14 Lactobacillus rhamnosus GR1 were grown in BHI broth in anaerobic conditions at 37 C. Escherichia coli DH5a were grown in Luria broth containing the appropriate antibiotic.

Coculture experiment were carried out using two-compartment devises (kindly provided by Dr F. Chaucheyras-Durand, INRA Clermont-Ferrand/Theix, France). The two 30 ml compartments were separated by a 0.45 um mixed cellulose esters membrane (Millipore). The cocultures were grown in BHI at 37 C, with a very low shaking to improve the exchanges between the two compartments. L. reuteri RC14 or L. rhamnosus GR1 (5%, vol/vol innoculum from an overnight culture) were inoculated first in one of the compartment and grown for 5 h (OD 600 nm ˜0.1-0.15), before inoculating S. aureus (1%, vol/vol innoculum from an overnight culture) in the second compartment. The cocultures were then grown overnight, and then checked for cross-contamination across the membrane on selective agar media (Mannitol Salt for S.aureus and Ragosa for Lactobacillus sp.)

Supernatant of L. reuteri RC14 was prepared as following. After an overnight growth in BHI at 37 C (OD600 nm ˜0.4-0.6), the culture was centrifuged (10 min, 5000 g, 4 C) and the supernatant was then filter sterilized using 0.2 um filters.

2D-PAGE Analysis of S. aureus Cell Surface Associated Proteins

The cell-surface proteins were extracted from S. aureus Newman similar to the procedures described by Hermann et al. (2000). Cultures (30 ml) were harvested by centrifugation at late exponential phase, and the cells were washed in 50 mM Tris-HCl (pH 7.5). The final pellet was then resuspended in 2 ml of Sarcosyl buffer (50 mM Tris-HCl [pH 7.5], 150 mM NaCl, 1 mM MgCl₂, and 2% [wt/vol] N-lauroyl-sarcosine). After incubation on ice for 20 min, the cell suspensions were then centrifuged (10,000×g for 10 min at 4° C.). The supernatant was recovered and stored at −80° C., prior to analysis.

For 2D-PAGE, the Sarcosyl-extracted proteins were first precipitated with ‘Perfect Focus’ (Geno Technology, St Louis, Mo.) following the manufacturer's specifications. The precipitates were then resuspended in 200 μl of a rehydration buffer containing 9 M urea, 4% (wt/vol) CHAPS, 0.5% (vol/vol) Biolytes (3-10, BioRad Laboratories, Hercules, Calif.) and 20 mM dithiothreitol, and left at room temperature for 1 h with occasional mixing. Insoluble materials were removed by centrifugation (16,000× g for 1 h at 15° C.). The protein concentration of each sample was determined by a modified Bradford procedure (BioRad Laboratories, Hercules, Calif.). Aliquots of the solubilized proteins (6 tg for analytical gels and up to 200 μg for preparative gels) were then applied to Immobiline IPG strips (7 cm, pH range 4-7, BioRad Laboratories, Hercules, Calif.). The strips were rehydrated overnight at 50V in an IEF cell (BioRad Laboratories, Hercules, Calif.), then isoelectric focusing was performed using the following steps: 200 volts for 100 VH, 500 volts for 250 VH, 1,000 volts for 500 VH, and 8,000 volts for 8000 VH. After focusing, the strips were immersed in an equilibration buffer containing 6 M urea, 2% (wt/vol) SDS, 50 mM Tris-HCl (pH 8.8), 30% (vol/vol) glycerol and 65 mM dithiothreitol. After 15 min, the strips were placed in the same buffer except dithiothreitol was replaced with 135 mM iodoacetamide, and then left for an additional 15 min. The second dimension electrophoresis was then performed using Mini-Protean III electrophoresis units (BioRad Laboratories, Hercules, Calif.) according to manufacturers specifications. The stacking gels and separating gels used were 4% T and 1 0% T, respectively. Following electrophoresis, the analytical gels were stained with SYPRO Ruby stain (BioRad Laboratories, Hercules, Calif.) following the recommendation of the manufacturer. Preparative gels were stained with Coomassie Blue R-250, with a staining step of 1 h in 50% methanol, 10% acetic acid with 0.1% Brilliant Blue R250, and a destaining step in 50% methanol, 10% acetic acid until the background became clear (about 2-3 h). Both types of gels were stored in 5% acetic acid at 4 C.

The 2D protein profiles were analyzed using Phoretix-2D (version 5.1) software (Nonlinear Dynamics Limited, Newcastle upon Tyne, UK). Relative volumes were estimated by calculating the ratio of the volume of a spot to the volume of the spots from the entire gel. Results are the means of at least three independent experiments. In the studies of differences between two conditions, proteins were considered to be induced or repressed if the mean relative volume for an individual protein was at least 2-fold higher or lower than that for the control.

Identification of the Proteins

Peptide mass fingerprints were obtained for the proteins using facilities provided by UWO Biological Mass Spectrometry Laboratory at the University of Western Ontario (London, Canada). The proteins were digested with trypsin, following the protocol provided by the facilities, available on the web site (http://www.biochem.uwo.ca/wits/bmslibmslhome.html

Briefly, the excised gel pieces were washed and dried in acetonitrile, and the proteins were subjected to reduction/alkylation by using dithiotreitol (10 mM) and iodoacetamide (55 mM), respectively. After several washing steps in 100 mM ammonium bicarbonate and dehydration in acetonitrile, a solution of trypsin (15 ng/ul) was added to each gel piece, and the digestion was performed overnight at 37 C. The digested fragments were then recovered with a solution of acetonitrile:formic acid (50:5 vol/vol). The peptide preparation was then dried in a speed vaccum and the dried peptides were stored at −80 C.

The peptide mixture was diluted 1:1 with α-cyano-4-hydroxycinamic acid (as a matrix). The MALDI-TOF (matrix assisted laser desorption/ionization time-of-flight) analysis of the samples was performed using a Bruker Reflex III (Bruker, Breman, Germany) mass spectrometer operated in linear, positive ion mode with the N₂ laser. Mascott databse search

The peptide sequence data were also used as query sequences in BLASTx searches of the S. aureus unfinished genome sequence data available via The Institute for Genomic Research's (TIGR) website (http://www.tigr.org). The open reading frames (ORF) within the contigs retrieved by the BLAST search were identified and their theoretical tryptic peptide fingerprints were determined via the Expasy web site (http://www.expasy.ch), and compared with the peptide mass fingerprints obtained by MALDI-TOF analysis of the proteins.

The characteristics of the SA (surface associated) proteins identified by the experiments are described below.

SA1 is homologous to an EXOTOXIN (S. aureus COL genome). It belongs to Superantigen family and located on a pathogenicity island. SA1 involved in the infection process. From the proteomic experiment, SA1 ‘disappears’ when S. aureus is in co-culture with Lactobacillus.

MS (Mass Spectrometry) data (sequence tags, mass fingerprints) and databases (SwissProt, genome of S. aureus strains) showed that SA2 was homologous to a cysteine synthase and involves amino acid synthesis pathway. SA2 may also relates to response to stress. SA2 decreased when S. aureus was in co-culture with Lactobacillus. SA3 is homologous to a superoxide dismutase. SA also involves reduction of oxygen radicals and response to hydrogen peroxide production by Lactobacillus. SA3 increased when S. aureus was in co-culture with Lactobacillus.

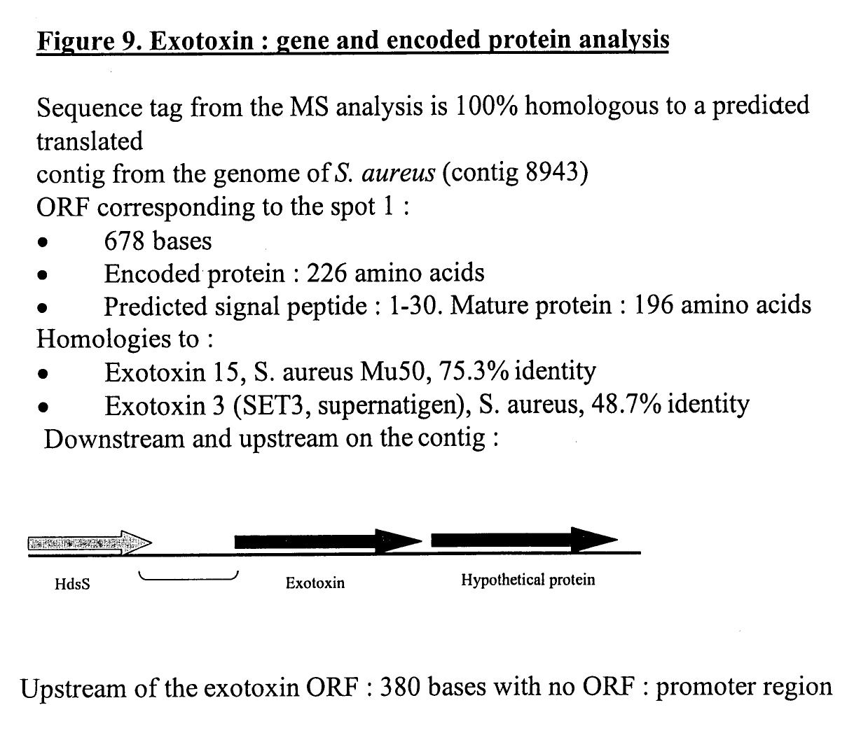

FIG. 9 shows that a gene and protein analysis demonstrated that SA1 is homologous to Exotoxin.

### EXAMPLE 5

In order to verify that the genes encoding the exotoxin are turned off by the Lactobacilli signaling molecules, a gfp-lux ABCDE reporter operon (Qazi, et al. Infection and Immunity 69:7074-82, 2001) was employed, which essentially allows detection of fluorescence when the exotoxin gene is turned on in s. aureus. Using this method, it was confirmed that indeed the exotoxin gene is turned off by Lactobacilli RC-14 and not by Lactobacilli GR-1 or other controls. Such result shows that the event is specific and novel.

DNA Preparation and Cloning in E. coli

Routine DNA methods were performed as described by Sambrook et al. Molecular Cloning (CSHL Press, 1989). Restriction endonucleases and DNA-modifying enzymes were purchased from Invitrogen and New Englands Biolabs (Mississauga, Ontario, Canada). Plasmid pSB2034, harbouring lux and gfp genes were used. Plasmid DNA was isolated using the QIA prep plasmid spin columns (Qiagen Inc., Santa Clarita, Calif.). Extraction of digested plasmid DNA from agarose gels was carried out using either QIAquick gel extraction kit or QIAEXII gel extraction kit (both from QIAGEN). Polymerase chain reaction (PCR) amplifications were all performed using the Platinium Taq (Invitrogen), according to the recommendations of the manufacturer. All oligonucleotides, containing or not restriction sites at their 5′ends, were purchased from Invitrogen. Digested PCR products were purified using the QIAquick PCR purification kit (Qiagen Inc., Santa Clarita, Calif.).

Construction of a lux gfp Reporter Gene System

The promoter of the exotoxin-lux, gfp fusion was constructed by PCR Amplification of a 384-bp DNA fragment corresponding to the untranslated 5′ end of the exotoxin gene (bases −1 to −385). The PCR product was cloned as an EcoRI/XmaI fragment into the unique EcoRI/XmaI site of pSB2034. The resulting plasmid was confirmed to contain the exotoxin promoter region directly upstream of the vector-borne lux, gfp, thus creating a transcriptional fusion, by sequencing the promoter region and the insertion sites (Sequencing Facility, John P. Robarts Research Institute, London, ON).

Lux/gfp fusion plasmid was recovered from E. coli and introduced into S. aureus RN4220 by electroporation, by selecting on TSB agar plates containing 10 ug/ml chloramphenicol. Phage transduction, using 80a, was used to transfer the fusion plasmid into S. aureus Newman. S. aureus Newman harbouring the plasmid was selected on TSB plates containing 50 ug/ml chroramphenical, and by checking the expression of the gfp by microscopic observations.

1. Cloning of the Exotoxin Promoter into E. coli DH5′Υ

Cloning of the exotoxin promoter (PROM) into plasmids pSB2033 and pSB2034

PCR of the Exotoxin Promoter:

Primers: PROMf (TAACTTTGATAAATACATAG, base−385; SEQ ID NO: 1) and PROMr (TTAAACCCTCGTATCTTAA, base−1; SEQ ID NO: 2). PROMf got an EcoR1 restriction site and PROMr got a XmaI restriction site.

DNA was extracted from S. aureus using Instagene Matrix, following the manufacturer's recommendations (BioRad)
PCR reaction: 5 ul DNA template
   5 ul of 10× buffer
   1 ul of each primers (50 pmoles/ul)
   2.5 ul of MgCl₂ (50 mM)
   2 ul of dNTP mix (5 mM)
   1 ul of Platinium Taq (5 U/ul)
PCR cycles: 1 cycle: 94 C 5 min
   30 cycles: 94 C 1 min
      55 C 1 min
      72 C 3 min
   1 cycle: 72 C 7min
      Digestion of Vector pSB2033/pSB2034 and of the PCR Product:

Vectors: pSB2033 and pSB2034. Each 12.5-kb plasmid contains both the gfp and lux genes for use as reporter genes. Ampicillin and chloramphenicol resistance.

| | |
|---|---|
| DNA | 15 ul |
| ECORI | 1 ul |
| XmaI | 1 ul |
| ddH₂O | 4.5 ul |
| BSA 25X | 1 ul |
| Buffer 10X | 2.5 ul |

Incubate 2 h at 37 C
Purification of Insert and Vector:
Purify inserts (PROM): Used Quiaquick gel purification kit according to manufacturers directions. Elution in 30 ul buffer.
Purification of vector: (pSB2033/pSB2034): Used QiaExII elution kit according to manufacturers directions. Elution in 30 ul buffer.
   Ligations:

Ligations were performed using the exotoxin promoter (PROM) and bot the pSB2033 and pSB2034 digested vectors to create a vector containing the GFP and LUX operons under the control of PROM.

Recipe:

1 ul of ligase (added last, kept on ice)

3 ul of vector (pSB2033 or pSB2034)

12 ul of insert (PROM)

4 ul of 5× ligation buffer

*The ligation reaction was left for 1 hour in a room temperature water bath and then subsequently left overnight at 4 C. The following morning, 1 ul of ligase was added to the ligation mixture and left at room temperature for 1-2 hours. The ligation was checked on a 1% agarose gel

Transformation of E.coli DH5′Υ with the pSB2034/PROM and pSB2033/PROM Ligations:

E. coli DH5′Υ competent cells were used that were previously made competent Procedure:
1) Place frozen competent cells on ice from the −80 C freezer
2) Allow cells to thaw on ice in the eppendorf tubes
3) Add 5 ul of each ligation mixture to a different tube of competent DH5α (PROM/pSB2033 or PROM/pSB2033). For a positive control, add 1 ul of puc19 to a tube also.
4) Leave the competent cells/DNA mixtures on ice for 30 min
5) Place the eppendorf tubes containing the competent cells and DNA into a 42 C water bath for 90 s. Do not shake the tubes.
6) Transfer the tubes containing the competent cells/DNA to ice for 1-2 min.
7) Add 800 ul of room temp SOC medium to each tube.
8) Transfer the tubes to a 37 C shaking incubator for 45 min.
9) Plate 100 ul and “the rest” of each transformation on LB 100Ap plates
   PCR on the E. coli DH5′Υ/PROM/pSB2034 Colonies:

(1) PCR was performed directly on the transformed colonies to confirm positive clones.
(1) Place colonies into eppendorf tubes containing 50 ul of ddH₂O
(2) Boil the colonies in a water bath for 5 min

(3) Use 5 ul of the product for the PCR reaction

RECIPE/PER COLONY:

| | | |
|---|---|---|
| DNA | 5 | ul |
| 10X buffer | 5 | ul |
| PROMR primer | 1 | ul |
| PROMF primer | 1 | ul |
| MgCl₂ | 2.5 | ul |
| 5X dNTPs | 2 | ul |
| Taq polymerase | 1 | ul (add last) |
| Filtered ddH₂O | 32.5 | ul |

PCR PROGRAM: (30 cycles)

| | | | | |
|---|---|---|---|---|
| 1 | cycle | 95 C. | 5 | min |
| 30 | cycles | 95 C. | 1 | min |
| | | 55 C. | 30 | s |
| | | 72 C. | 45 | s |
| 1 | cycle | 72 C. | 10 | min |

Digestion on DH5′Υ/PROM/pSB2034 PCR Products (Should Cut within the PROM Insert):

*Used to confirm positive clones

1) PCR products were first cleaned using the Qiagen PCR purification kit. The PCR products were eluted in 40 ul of elution buffer.

2) Digestion with HincII (Promega Buffer B) and digestiong with DpnI (Invitrogen Buffer 4).

Recipe for Digestion on PCR Products:

10 ul of PCR product

2 ul of appropriate buffer

1 ul of appropriate enzyme

0.8 ul of 25× BSA

6.2 ul ddH2O

**allow the digestion to proceed for 2 hr in a 37 C water bath

Mini-Preps and Digestions on the DH5α/PROM/pSB2034 Colonies (Cuts Around Insert):

*Used to further confirm positive clones

1) Mini-preps were performed using the Qiagen spin miniprep protocol as outlined by the manufacturer

2) Each miniprep was digested with ECORI and then ECOR1/XmaI (cuts insert out)

RECIPE FOR DIGESTIONS WITH ECOR I:

| | |
|---|---|
| DNA | 4 ul |
| EcoR I | 1 ul |
| ddH₂O | 16.5 ul |
| Buffer bio4 | 2.5 ul |
| BSA 25X | 1 ul |

RECIPE FOR DIGESTIONS WITH ECORI/XmaI:

| | |
|---|---|
| DNA | 4 ul |
| EcoR I | 1 ul |
| XmaI | 1 ul |
| ddH₂O | 15.5 ul |
| BSA 25X | 1 ul |

**Allow digestion reactions to proceed for 2 hr in a 37 C water bath

**Run the entire 25 ul on a 1% agarose gel to confirm results

Sent Samples for Sequencing

** To confirm positive clones

2. Transformation of S. aureus RN4420 with PROM/pSB2034

This step was required so that S. aureus RN4420 can methylate the plasmid DNA. This allowed the subsequent transformation of the gene reporter vector harboring the exotoxin promoter into S. aureus Newman.

Electrocompetent S. aureus RN4420:

This procedure must be done cold and sterile (ie. on ice by a flame)

1) Prepare an overnight culture of RN4420 in 5 ml of TSB

2) Innoculate 100 ml of TSB with the 5 ml overnight culture and incubate in a 37 C shaking incubator until an O.D. of approx. 0.5 is reached.

3) Allow the cells to sit on ice for 10 min.

4) Harvest the cells at 4 C at 5000 rpm for 10 min.

5) Wash the cells twice using 10 ml of ICE COLD 500 mM filter-sterilized sucrose

6) Re-suspend the cells in 1 ml of ICE COLD 500 mM sucrose

7) Allow the cells to sit on ice for 20 min.

8) Harvest the cells at 4 C in a microcentrifuge tube and re-suspend in 1 ml of ICE COLD 500 mM sucrose.

9) Aliquot 60 ul into pre-chilled eppendorf tubes and freeze at −80 C.

Electroporation of S. aureus RN4420:

*Used to permit the uptake of pSB2034/PROM by the electrocompetent RN4420

a. Thawed 60 ul of electrocompetent RN4420 on ice.

b. Transfered 1 ul or 5 ul of pSB2034/PROM to individual eppendorf tubes

c. Left on ice for 30-60 s

d. Transferred the contents of the eppendorf tubes into cold electroporation cuvettes (from the fridge).

e. Pulsed the electrocompetent cells: 2.4 kV, 25 uF, 100 Ohms (or 200 Ohms).

f. Added 1 ml of TSB quickly after pulse.

g. Incubated the cells at 37 C in their cuvettes for 1 hour, not shaking.

h. Added chloramphenicol to the cuvette to a final concentration of 0.2 ug/ml.

i. Incubated at 37 C for one hour (not shaking).

j. Plated 200 ul on TSB 10 Cm.

3. Preparation of the Infective Lysate from S. aureus RN4420

* Put the plasmid of interest from RN4420 into phage

* To be done on overnight cultures of RN4420 harboring the plasmid of interest and grown in TSB10 Cm.

* Need at least 1.5 ml of each culture

* Phage was stored in the fridge

* Be extremely sterile: new eppendorf tubes, new pipette tips, clean desk with ethanol prior to this procedure, use flame.

1.Diluted an overnight culture of RN4420 harboring the pSB2034/PROM plasmid in ½ in TSB 2.5 mM CaCl₂ (500 ul:500 ul) in separate tubes per strain (ie testing #10 and #12).

2.Incubated for 10 min at 3 C

3.Added 100 ul of phage to tube #1

10 ul of phage to tube #2

1 ul of phage to tube #3

(This assumes that the phage is at a reasonably high titer)

4. Incubated the tubes at 37 C for 15 min

5. Added each mixture to a separate 20 ml solution of TSB with 2.5 mM CaCl₂ (a clear conical tube).

6. Incubated at 37 C on a slow shaker until lysis occurs (100 ul should lyse first, goes clear). The lysis step usually takes around 4-5 hr.

7. Spinned out debris (7000 rpm/10 min). A pellet was not visible, just took 15 ml from the top and filter sterilized that part of the supernatant (0.45 micron).

8. Stored supernatant at 4 C.

4. Transduction of S. aureus Newman with the Infective Lysate

** Need an overnight culture of S. aureus Newman grown in TSB 2.5 mM CaCl₂

** Need 2.5 mM Na. citrate 10 Cm TSB plates
I. Grew S. aureus Newman culture overnight to an O.D. of 0.5 to 0.75 in 20 ml of TSB 2.5 mM CaCl₂.
II. Aliquoted 1 ml of culture supernatant into as many eppendorf tubes as the infectious lysates to be tested. (i.e., if testing infective lysates from strain #12 and #10 from RN4420, 2 tubes of 1 ml of Newman culture would be needed).
III. Spinned down S. aureus Newman cultures in microcentrifuge at top speed for 2 min.
IV. Resuspended the pellet in 500 ul of TSB 2.5 mM CaCl₂.

V.

Aliquoted (5 tubes/clone)

| TUBE # | CELLS | INFECTIVE LYSATE | |
|---|---|---|---|
| 1 | 100 ul | 0 | ul (negative control) |
| 2 | 100 ul | 0.1 | ul |
| 3 | 100 ul | 1 | ul |
| 4 | 100 ul | 10 | ul |
| 5 | 100 ul | 100 | ul |

VI. Incubated the tubes above for 20 min, 37 C in a non-shaking incubator.
VII. Added 1 ml of TSB with 3.0 mM Na-citrate to each tube.
VIII. Incubated for at least 1 hour at 37 C in a non-shaking incubator.
IX. Pelleted the tubes in a microcentrifuge (top speed, 2 min) and removed all but 100 ul of the supernatant.

X. Resuspended the pellet in the 100 ul of supernatant and plated the 100 ul of culture on 2.5 mM Na-citrate 10 Cm plates.

TABLE 4

| | | | Cocult | Cocult | | | | | |
|---|---|---|---|---|---|---|---|---|---|
| | | | RC14/ | GR1/ | | | | | |
| | Expe | Expe | Mono | Mono | | | Theor | Theor | |
| | MW | pI | cult | cult | Homologous | % | MW | pI | Sequence tags |
| 1 | 22 | 5.9 | | → | Exotoxin | Coverage | 22.3 | 6.0 | SGNTASIGGITK |
| | | | | | | 64% | | | (SEQ ID NO: 3) |
| 2 | 30 | 5.4 | | → | Cysteine | Coverage | 33.0 | 5.5 | TIDAFLAGVGTG |
| | | | | | synthase | 21.29% | | | GTLSGVGK (SEQ |
| | | | | | | | | | ID NO: 4) |
| 3 | 22 | 5.1 | ↑ | ↑ | Superoxide | Coverage | 22.7 | 5.2 | LNAAVEGTDLES |
| | | | | | dismutase | 17.08% | | | KSIEEIVANLDSV |
| | | | | | | | | | PANIQTAVR |
| | | | | | | | | | (SEQ ID NO: 5) |
| 5 | 22 | 5.4 | | → | Hypothetical | Coverage | 22.3 | 5.3 | LTLQVVSIDEQGK |
| | | | | | protein | 16.58% | | | (SEQ ID NO: 6) |
| 6 | 26 | 5.6 | ↑ | → | 30S | Coverage | 29.1 | 5.4 | AGQFYINQR |
| | | | | | ribosomal | 7.84% | | | (SEQ ID NO: 7) |
| | | | | | protein | | | | |
| 7 | 60 | 5.8 | ↑ | ↑ | Formyltetrahydrofolate | Coverage | 59.8 | 5.7 | IVTEIYGGSK |
| | | | | | synthetase | 4.80% | | | (SEQ ID NO: 8) |
| 9 | 23 | 4.8 | ↑ | → | ABC | Coverage | 29.1 | 4.7 | YLNEGFSGGEK |
| | | | | | transporter | 7.90% | | | (SEQ ID NO: 10) |
| | | | | | (ATP | | | | |
| | | | | | binding prot) | | | | |
| | | | | | (Bacillus | | | | |
| | | | | | halodurans) | | | | |

TABLE 5

| | | Homologous protein | | MW (kDa) | pI | |
|---|---|---|---|---|---|---|
| | | (organism, accession | | Measured/ | Measured/ | Coverage |
| Spot # | n-fold | number) | Sequence tags | Therotical | Therotical | (%) |
| 1 | −5.6 | Exotoxin 15 | SGNTASIGGITK | 24/22.3 | 5.9/6.0 | 64 |
| | | (Staphylococcus aureus | (SEQ ID NO: 11) | | | |
| | | Mu50, Q99WG9) | | | | |
| 2 | −2.1 | Cysteine synthase | TIDAFLAGVGT | 34/33 | 5.4/5.5 | 21.29 |
| | | (Staphylococcus aureus | GGTLSGVGK | | | |
| | | Mu50, NP_371037) | (SEQ ID NO: 12) | | | |
| 3 | +2.1 | Superoxide dismutase | LNAAVEGTDLE | | | |
| | | (Staphylococcus aureus | SKSIEEIVANLD | 25/22.7 | 5.1/5.2 | 17.08 |
| | | Mu50, NP_372077) | SVPANIQTAVR | | | |
| | | | (SEQ ID NO: 13) | | | |
| 5 | −2.6 | Hypothetical protein | LTLQVVSIDEQ | 24/22.3 | 5.4/5.3 | 16.58 |
| | | (Staphylococcus aureus | GK (SEQ ID NO: | | | |
| | | Mu50, NP_372378) | 14) | | | |
| 6 | +2.1 | 30S ribosomal protein | AGQFYINQR | 34/29.1 | 5.6/5.4 | 7.84 |
| | | (Staphylococcus aureus | (SEQ ID NO: 15) | | | |
| | | Mu50, NP_371780) | | | | |
| 7 | +8.6 | Formyltetrahydrofolate | IVTEIYGGSK | 60/59.8 | 5.8/5.7 | 4.80 |
| | | synthetase | (SEQ ID NO: 16) | | | |
| | | (Staphylococcus aureus | | | | |
| | | Mu50, NP_372256) | | | | |
| 9 | −2.4 | ABC transporter (ATP | YLNEGFSGGEK | 28/29.1 | 4.8/4.7 | 7.90 |
| | | binding prot) | (SEQ ID NO: 17) | | | |
| | | (Bacillus halodurans, | | | | |
| | | NP_244338) | | | | |

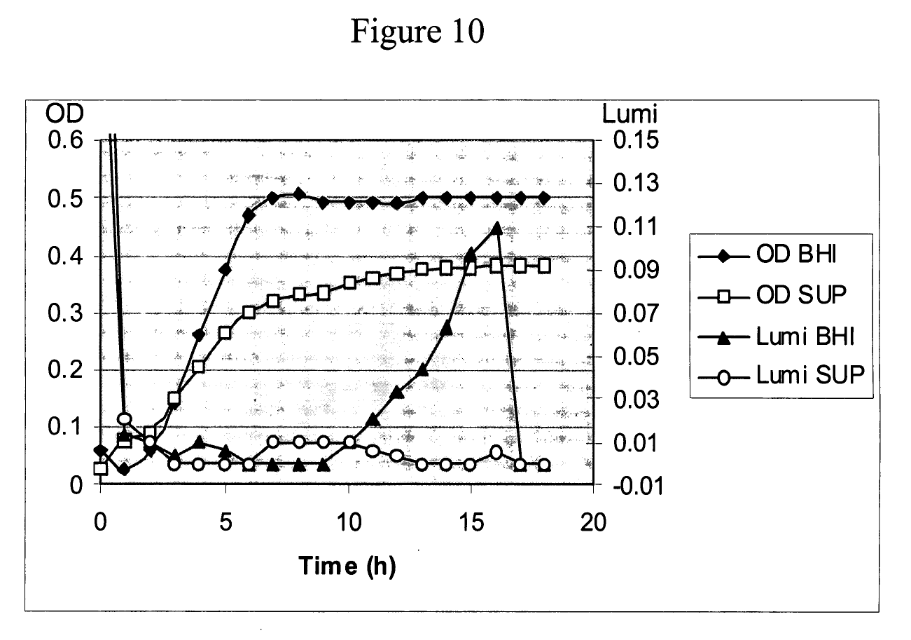

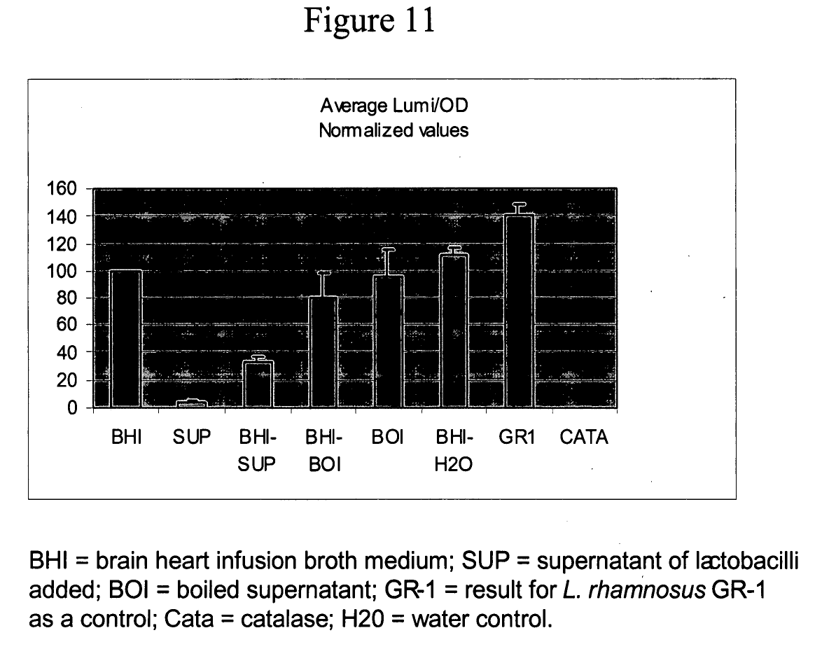

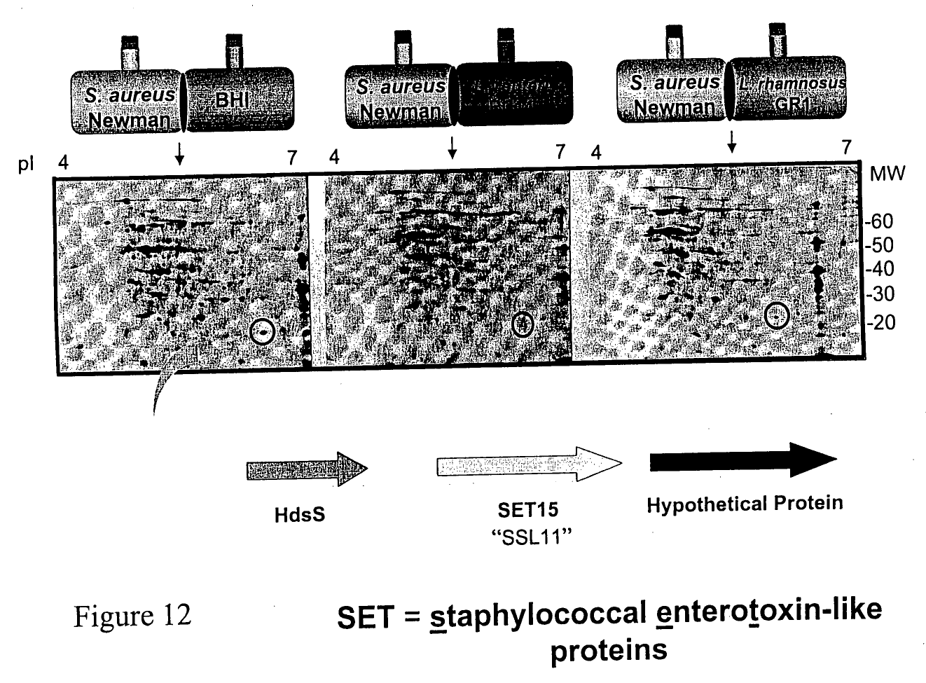

These experiments, as illustrated in FIGS. 10 and 11, clearly showed that Lactobacilli reduced and all but eliminated expression of exotoxin in S. aureus.

### EXAMPLE 6

A further series of experiments were performed which resulted in the demonstration of a much broader spectrum of anti-virulence activity by Lactobacilli against pathogens.

The co-cultures experiments were set up. The co-cultures were carried out using two compartment devises as described in Example 4 or similar devices commercially available. In control experiments were carried out with S. aureus Newman grown in BHI medium in one compartment and BHI medium only in the other compartment. The two co-culture experiments were carried out with S. aureus Newman grown in BHI medium in one compartment and either L. reuteri RC-14 or L. rhamnosus GR1 grown in the other compartment. Staphylococcal enterotoxin-like (SET) proteins were investigated.

Set1 stimulates cytokine release from PBMCs. Crystal structures of SET3 and SET6 have been resolved. SET15 is structurally related to S. aureus and S. pyogenes superantigens. SET15 is located on S. aureus pathogenicity island 2 and is found in all S. aureus strains sequenced. The results in the present invention showed that when S. aureus was grown in co-culture with L. reuteri RC-14, the expression of SET15 was significantly decreased.

P3 is a promoter of the agr system in S. aureus. Activation of P3 increases expression of exoproteins.

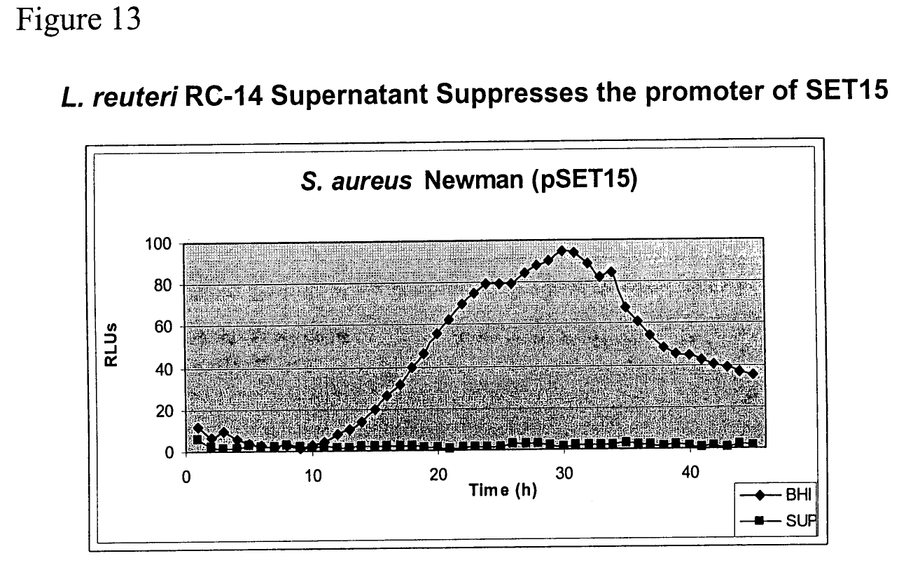

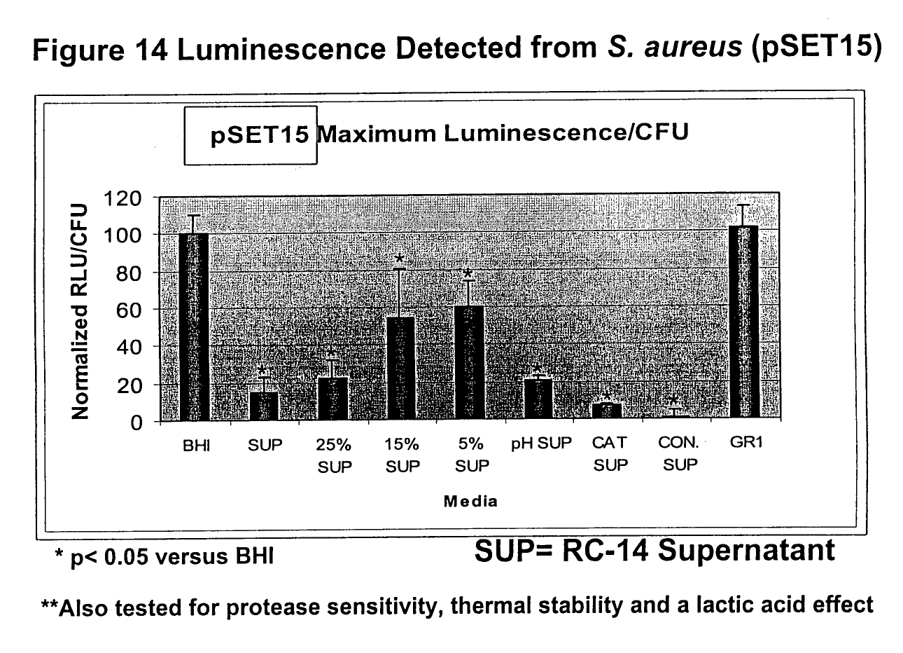

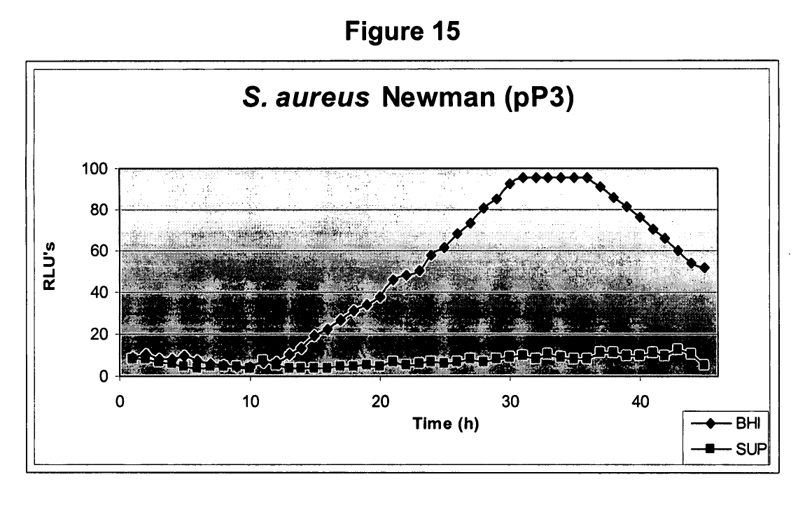

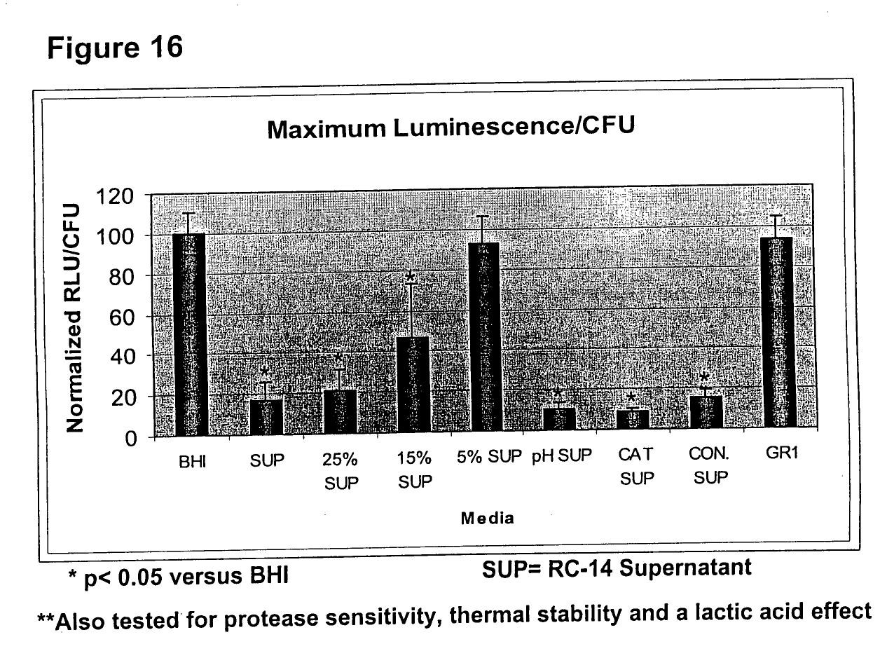

Plasmids pSET15 and pP3 were constructed in which SET15 promoter and P3 were each inserted upstream of GFP and Luciferase. The co-culture result, as illustrated in FIGS. 13-16, showed that L. reuteri RC-14 supernatant suppresses the promoter of SET15.

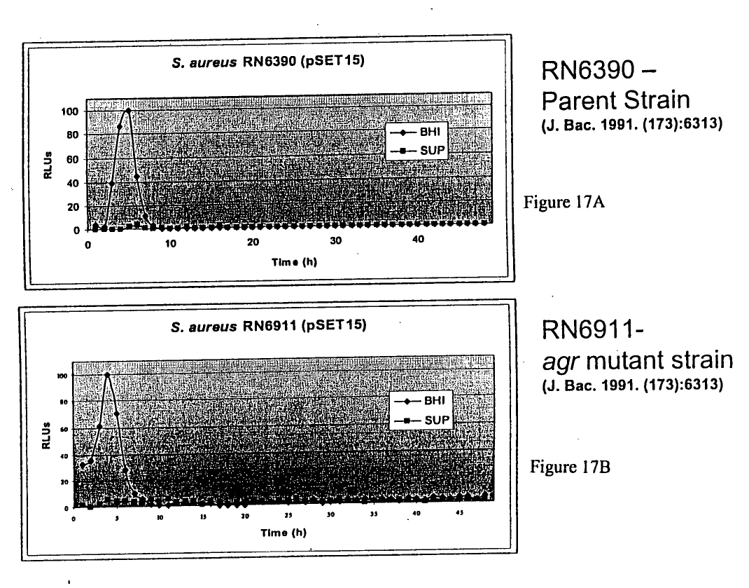

Both the SET15 promoter and P3 promoter were suppressed when S. aureus was grown in the presence of L. reuteri RC-14 supernatant. To investigate if the decrease in SET15 expression a result of a decrease in P3 activation, an agr mutant strain was employed, which lacks P3 promoter activity. The result demonstrated that, as shown in FIG. 17, SET15 expression or the suppression of the SET15 promoter is independent of the agr pathway.

### EXAMPLE 7

Further identification of the signal molecules from Lactobacillus was performed. HPLC fractions that contain the isolated active compound were collected, which were characterized.

HPLC was performed using an Agilent 1100 HPLC apparatus equipped with a 25-cm C8 column (Agilent). The fraction was loaded onto the column in 0.1% trifluoroacetic acid and eluted in a gradient of 2-80% acetonitrile in 0.1% trifluoroacetic acid over a period of 30 ml. The results showed that Fraction 63 contained the active compound. MS analysis was performed on a Micromass Quattro Micro mass spectrometer equipped with a Z-spray source operating in the positive ion mode. Raw electrospray ionization mass spectrometry (ESI-MS) data for Fractions 62, 63 and 64 showed that Fractions 63 and 64 contained the active compound while fraction 62 was inactive.

### EXAMPLE 8

### Effects of Lactobacillus jensenii 25258 Spent Culture Supernatant on Escherichia coli C1212 Virulence Factor Expression

A further example of how bacterial molecules can be used to affect pathogenic bacteria came from experiments using Lactobacillus jensenii PC1. This was the first declaration of activity in this strain, illustrating that the concept of signaling and down regulation of E. coli virulence factors extends beyond strain RC-14. These experiments were performed using promoters that specifically indicate whether the Fim A, OmpA, Omp X and Pap A genes are turned off or down regulated.

Experimental Details:

Methods

Lactobacillus strains were grown for 48 hrs at 37 C in modified Mann Rogosa Sharpe (MRS) media containing 0.06 mM FeSO₄ and 2% glycerol and devoid of dextrose and beef extract. Supernatants were filter sterilized through a 0.2 mm membrane and stored at 4 C until use.

Reporter constructs were generated containing the promoter regions of four Escherichia coli C1 212 virulence factor genes placed immediately upstream of the luciferase (lux) expression operon contained in vector pSB2034. The gene promoters used were those of Outer membrane protein X (OmpX), Outer membrane protein A (OmpA), the FimA subunit of Type 1 fimbriae and the PapA subunit of P fimbriae. This was accomplished by PCR amplifying the promoter regions from E. coli C1212 gDNA using specific oligonucleotide primers containing the restriction sites EcoRI (Forward primer) and XmaI (Reverse primer). These amplicons and pSB2034 were digested sequentially with the aforementioned restriction enzymes and ligated together to generate the four reporter constructs. The resulting vectors were sequence verified and transformed into E. coli C1212 via electroporation. Transformants were selected based upon highest total luminescence versus optical density at 575 nm during log phase growth.

Lactobacillus spent culture supernatants (SCS) were tested for the ability to affect the growth and virulence factor expression of E. coli C1212 as follows: 1.5 mL cultures were generated for each test condition containing 375 mL (25%) 4× modified MRS and 1.125 mL (75%) Lactobacillus SCS or control solution. Each culture was inoculated with 1×105 colony forming units (CFUS) of the respective E. coli C1212 reporter transformant and the 1.5 mL aliquoted (225 mL/well) into triplicate wells of two 96 well plates. Both plates were incubated at 37 C for 24 hrs with no shaking. One plate was monitored for luminescence while the second for growth (OD 575 nm) and the results expressed at each time point as total luminescence versus optical density.

Results

Of the ten Lactobacillus strains tested, only L. jensenii PC 1 strongly inhibited the luminescence expression of all four virulence factor reporter constructs and did not reduce the maximal growth or growth rates of the E. coli C1212 transformants. On the contrary, L. jensenii PC 1 SCS increased both growth parameters in all four promoter reporter clones.

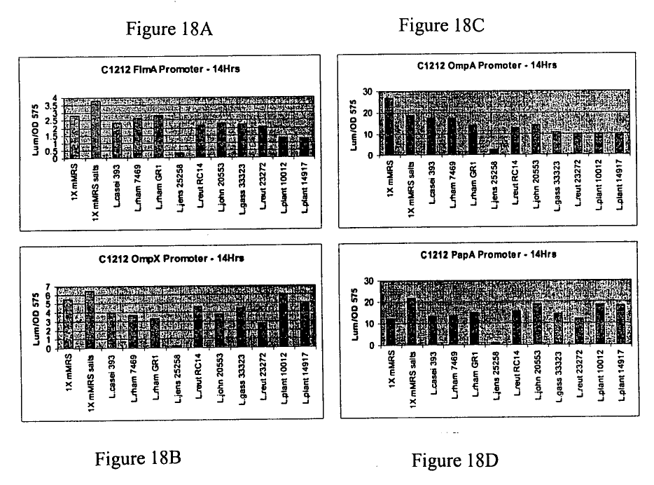

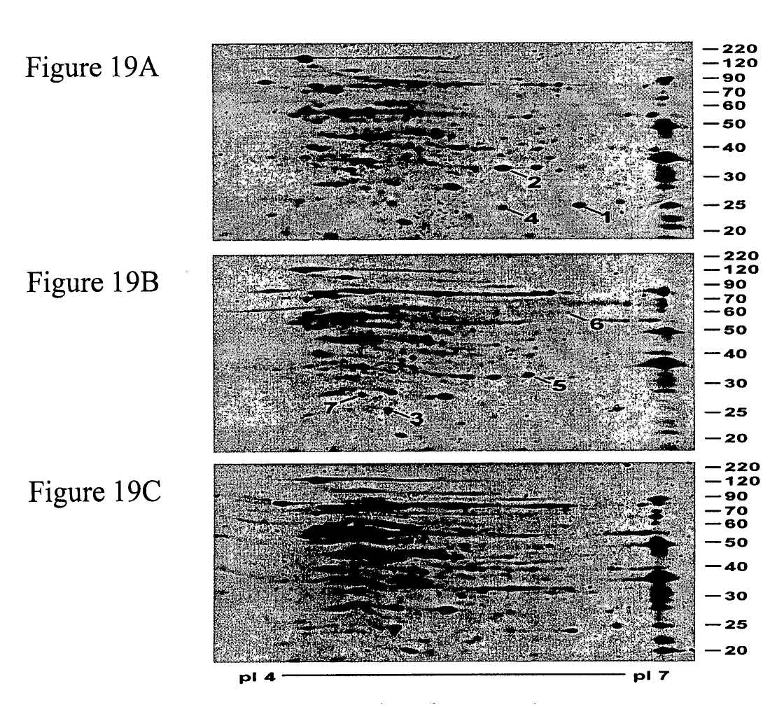

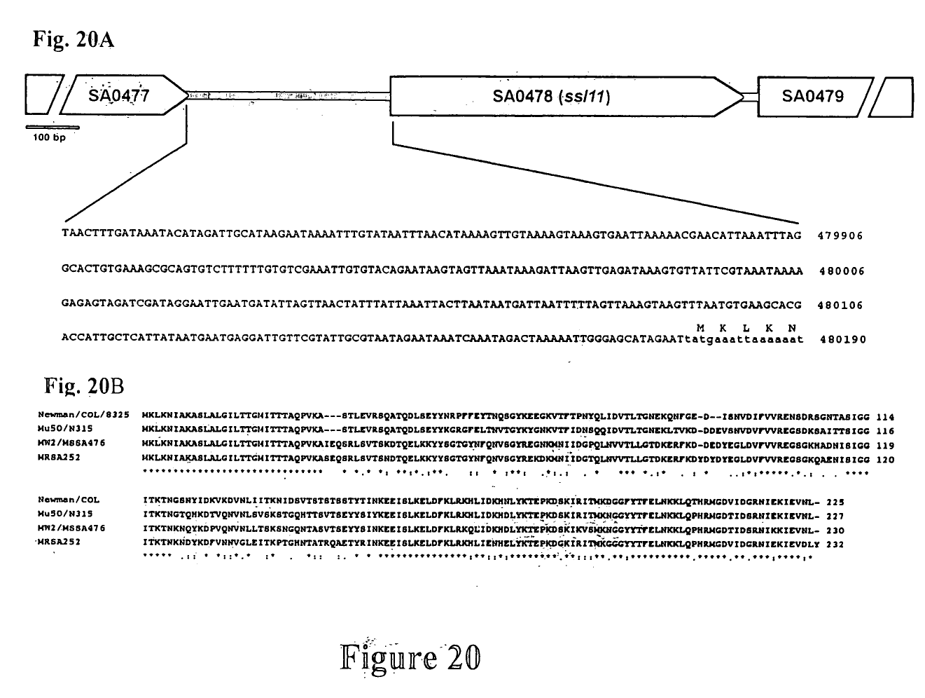

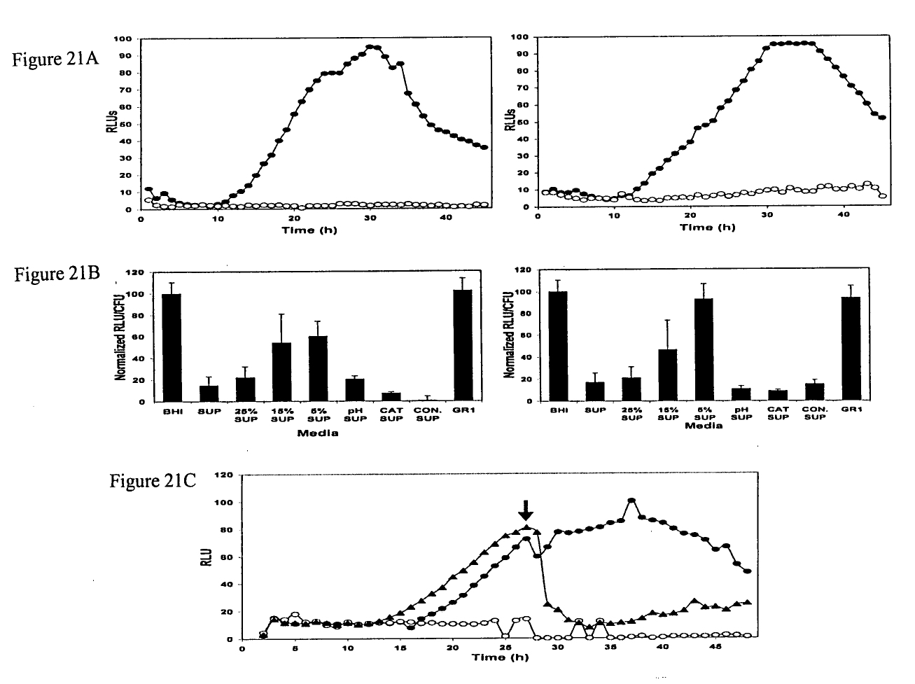

Since the total luminescence observed at the 14 hour timepoint represents the approximate maximal value for all four reporter clones over a 24 hour experiment, this value was chosen for comparison between the different Lactobacillus supernatants and media controls. L. jensenii PC1 SCS reduced the luminescence per OD 575 by 89.4, 95.4, 90.1 and 91.9% for FimA, OmpX, OmpA and PapA, respectively, compared to media alone. Simultaneously, the total growth of the aforementioned clones was increased by 83, 85, 154 and 93%, respectively, at 14 hours. The result is illustrated in FIG. 18.

### EXAMPLE 9

Table 6 below illustrates the result of an experiment performed by Dr. David Mack. As shown in Table 6, Lactobacillus RC-14's activity against staphylococcus was not due to the same signal that Dr. Mack discovered from L. plantatum 299v (positive control), as the signal was not produced by Lactobacillus RC-14. The present example demonstrates that Lactobacillus RC-14's anti-staphylococcus activity is not caused by mucin. This activity is not the same as the acid effect that kills viruses (see Cadieux et al. 2002). See also Example 8 above, which proves that the supernatant, in which the signalling molecules are present, did not reduce pathogen growth as lactic acid and bacteriocins do.

TABLE 6
MUC2 Data

| Group | n | Mean | SE |
|---|---|---|---|
| Cell control | 4 | 1.00 | 0.00 |
| Positive control | 4 | 1.85 | 0.3 |
| Negative Control | 3 | 1.06 | 0.6 |
| L. rhamnosus GR-1 | 4 | 1.26 | 0.19 |
| L. reuteri RC-14 | 4 | 1.07 | 0.12 |

### EXAMPLE 10

Lactobacillus reuteri RC-14 was previously shown to inhibit Staphylococcus aureus infection in a rat surgical implant model. To investigate this mechanistically, communication events between these two bacterial species were examined. L. reuteri RC-14 and S. aureus Newman were grown in a co-culture apparatus that physically separates the two species, but allows for the passage of secreted compounds. Protein expression changes in S. aureus were analyzed using two-dimensional gel electrophoresis (2-DE) in response to co-culture with L. reuteri RC-14, media alone, or a control Lactobacillus strain. Proteins of interest were identified by mass spectrometry and one protein in particular, identified as staphylococcal superantigen-like protein 11 (SSL11), showed a dramatic decrease in expression only in response to growth with L. reuteri RC-14. Genetic reporters were used that placed both gfp and lux under the transcriptional control of either the SSL11 promoter, or the P3 promoter of the staphylococcal accessory gene regulator (agr) locus. The SSL11 reporter confirmed the is 2-DE results, and a decrease in P3 promoter activation was also observed in the presence of L. reuteri RC-14 supernatant. The data further show however, that the repression of the SSL11 promoter is independent of the staphylococcal agr pathway. These results suggest that L. reuteri RC-14 is able to decrease the virulence potential of S. aureus via cell-cell signalling molecules.

Bacterial Strains and Plasmids

Bacterial strains and plasmids used in this study are listed in Table 7. Escherichia coli was cultured in Luria Bertani broth (Difco Laboratories Inc, Detroit, Mich.). Strains of S. aureus were grown in Brain Heart Infusion (BHI) broth (Difco). Solid media were obtained by the addition of 1.5% (w/v) Bacto-agar (Difco). To test for α-haemolysis and β-haemolysis, S. aureus was plated on sheep blood agar plates (Becton, Dickinson and Company, Loveton Circle, Md.). S. aureus cultures were grown without aeration at 37° C., and E. coli cultures were grown aerobically at 37° C. Lactobacilli strains were grown anaerobically in BHI medium or Man-Ragosa-Sharpe (MRS) medium (Merck Frosst Canada Ltd., Kirkland, QC) as required. For plasmid selection, chloramphenicol was used at 10 μg/mL for S. aureus RN4220 and E. coli, and at 50 μg/mL with all other S. aureus strains. Ampicillin and kanamycin were used in E. coli at 100 μg/mL and 50 μg/mL, respectively. Mannitol Salt Agar (MSA) (Difco) and Ragosa (Difco) agar were used as selective media for S. aureus and lactobacilli strains, respectively. All reagents were made with water purified through a Milli-Q water purification system (Millipore, Mississauga, ON).

Preparation, Manipulation, and Analysis of DNA

Standard DNA manipulations were performed as described (Sambrook and Russell, 2001) using enzymes supplied from New England Biolabs (Pickering, ON, Canada) in accordance with the manufacturer's instructions. Oligonucleotides were obtained from Invitrogen and are described in Table 7. Polymerase chain reactions (PCRs) were performed in a Peltier Thermocycler (MJ Research, Miami, Fla., USA) in 50-μl reaction volumes with Taq DNA polyrnerase or PFX DNA polymerase (Invitrogen, Burlington, ON). PCR products were purified using the QIAquick PCR purification kit (Qiagen Inc., Mississauga, ON). All DNA sequencing was performed at the Sequencing Facility, John P. Robarts Research Institute, London, ON, Canada. Plasmids were introduced into S. aureus RN4220 before being transduced into S. aureus Newman using bacteriophage 80α, as described (Sebulsky et al., J Bacteriol 182: 4394-4400, 2000).

Co-Culture Experiments

Co-culture experiments were performed using a two-chamber device. Specifically, two 30 mL glass compartments were separated by a 0.45 μm mixed cellulose ester membrane (Millipore). The co-cultures were grown in BHI at 37° C., with slow shaking (˜30 rpm) to improve diffusion of small molecules between compartments. L. reuteri RC-14 or L. rhamnosus GR-1 (5% vol/vol inoculum) were inoculated first and grown to an optical density at 600 nm (OD₆₀₀) of 0.15 (˜5 h) before inoculating S. aureus Newman (1% vol/vol inoculum) in the second compartment. After overnight growth, the co-cultures were examined to ensure no cross-contamination occurred between compartments using selective agar media (MSA for S. aureus and Ragosa for lactobacilli) and the OD₆₀₀ was measured for each compartment at the end of the co-culture experiments.

Preparation of Lactobacillus reuteri RC-14 Supernatant

Cell-free supernatants were prepared by growing L. reuteri RC-14 in BHI to an OD₆₀₀ of 0.4-0.6. Cells were removed by centrifugation (5000×g for 10 min at 4° C.). The remaining ceil-free supernatant was filtered using 0.45 μm filters, and checked for sterility by plating an aliquot on MRS plates. For catalase-treated supernatant, catalase (Sigma, Oakville, ON) was added to L. reuteri RC-14 supernatant to a final concentration of 1000 U/mL. For pH-adjusted supernatant, the pH of the supernatant was adjusted to the pH of BHI media using 5 N NaOH. For boiled supernatant, the supernatant was boiled for 30 min, and the volume was adjusted to the original volume by the addition of sterile water. For concentrated supernatant, 1/175 dilution of 200-fold concentrated L. reuteri RC-14 supernatant was added to BHI medium. For protease treated supernatant, 0.5 mg/mL of pronase, proteinase K or trypsin (or a combination of all 3) was added to L. reuteri RC-14 supernatant according to manufacturer's specifications (Sigrna). All supernatant preparations were then filter sterilized and checked to ensure sterility. To test for a lactic acid effect, 20 mM, 50 mM or 100 mM of lactic acid at a pH of 2 or a pH of 7 were added to BHI medium.

Extraction of S. aureus Cell Wall-associated Proteins and 2-dimensional Gel Electrophoresis (2-DE).

Cell surface-associated proteins were extracted from S. aureus Newman essentially as described (Hermann et al., Electrophoresis 21: 654-659, 2000). Specifically, 30 mL cultures were harvested (5000×g for 10 min at 4° C.) at late exponential phase and subsequently washed in 50 mM Tris-HCl pH 7.5. The final pellet was then resuspended in 2 mL of Sarcosyl buffer (50 mM Tris-HCl, pH 7.5, 150 mM NaCl, 1 mM MgCl₂, and 2% [wt/vol] N-lauroyl-sarcosine), and incubated oil ice for 20 min. The cell wall suspensions were then centrifuged (10,000×g for 10 min at 4° C.) and the supernatant was recovered, aliquoted and stored at −80° C. prior to analysis.

For 2-DE, the Sarcosyl-extracted proteins were first precipitated with the “Perfect Focus Kit” (Geno Technology, St. Louis, Mo.) following the manufacturer's specifications. Precipitated proteins were resuspended in 200 μl of rehydration buffer (9M Urea, 4% [wt/vol] CHAPS, 0.5% [vol/vol] Biolytes [3-10, BioRad Laboratories, Mississauga, ON] and 20 mM dithiothreitol) and left at room temperature for 1 h with occasional mixing. Insoluble material was removed by centrifugation (16,000×g for 1 hr at 15° C.). The concentration of each protein sample was then determined by a modified Bradford procedure (BioRad). Aliquots of the solubilized proteins (6 μg for analytical gels and up to 200 μg for preparative gels) were then applied to immobilized IPG strips (7 cm, pH range 4-7; BioRad). The strips were rehydrated overnight at 50 V in an isoelectric focusing (IEF) cell (BioRad) and the following day first dimension IEF was carried out with increasing voltage (200 V for 100 VH, 500 V for 250 VH, 1000 V for 500 VH and 8000 V for 8000 VH). After IEF, the strips were immersed in an equilibrium buffer containing 6 M urea, 2% (wt/vol) SDS, 50 mM Tris-HCl pH 8.8, 30% (vol/vol) glycerol and 65 mM dithiothreitol. After 15 min, the strips were placed in the same buffer except dithiothreitol was replaced with 135 mM iodoacetamide, and then the strips were left for an additional 15 min. The second dimension SDS-PAGE was performed using the Mini-Protean III electrophoresis unit (BioRad). The stacking gels and separating gels used were 4% and 10%, respectively. Following electrophoresis, the analytical gels were stained with SYPRO Ruby (BioRad). Preparative gels were stained with Coomassie Blue R-250. Gel images were captured using an AlphaInnotech camera and the 2-DE protein profiles were analyzed using Phoretix-2D (version 5.1) software (Non-linear Dynamics Limited, Newcastle upon Tyne, UK). Relative volumes were estimated by calculating the ratio of the volume of a spot to the volume of the spots from the entire gel. Results are the means of at least three independent experiments. In experiments that compared two conditions, proteins were considered to be induced or repressed if the mean relative volume for an individual protein was at least 2-fold higher or lower than that for the control.

Identification of Proteins of Interest

Peptide mass fingerprints were obtained for the proteins using facilities provided by The Biological Mass Spectrometry Laboratory at the Dr. Don Rix Protein Identification Facility at The University of Western Ontario (London, Canada). The proteins were digested with trypsin, following the protocol provided by the facilities. Briefly, the excised gel pieces were washed and dried in acetonitrile, and the proteins were subjected to reduction/alkylation by using dithiothreitol (10 mM) and iodoacetamide (55 mM), respectively. After several washing steps in 100 mM ammonium bicarbonate and dehydration in acetonitrile, a solution of trypsin (15 ng/gl) was added to each gel piece, and the digestions was performed overnight at 37° C. The digested fragments were then recovered with a solution of acetonitrile:formic acid (50:5 vol/vol). The peptide preparation was then dried in a speed vacuum and the dried peptides were stored at −80° C. until needed. The peptide mixture was then diluted 1:1 with α-cyano-4-hydroxycinamic acid. MALDI-TOF-MS analysis of the samples was performed using a Reflex III (Bruker, Breman, Germany) operating in a linear, positive ion mode with the N₂ laser to obtain MS fingerprints and sequence tags. A Mascott Database search was then performed to identify proteins based on their pI, molecular weight, and mass fingerprint (only protein databases were searched). The peptide sequence data (Table 1) were also used as query sequences in BLASTx searches of the finished S. aureus genomes. The ORFs within the contigs retrieved by the BLAST search were identified and their theoretical tryptic peptide fingerprints were determined via the Expasy web site (http://www.expasy.ca), and compared with the peptide mass fingerprints obtained by MALDI-TOF-MS analysis of the proteins. For confirmation, the peptide sequence data were also used as query sequences in BLASTp searches of all completed S. aureus genomes available on the TIGR website.

Creation of the gfp/lux Gene Reporter Construct

pSB2034 is an E. coli /Gram-positive bacterial shuttle vector that contains both gfp and lux under the transcriptional control of the staphylococcal P3 promoter (Qazi et al., Microb Ecol 41: 301-309, 2001). The P_{SSL11}::gfp-lux fusion was constructed by PCR amplification of a 385-bp DNA fragment corresponding to the untranslated 5′ end of the ssl11 gene (bases −1 to −385) using primers P_{SSL11}::gfp/lux (forward) and P_{SSL11}::gfp/lux (reverse). The PCR product was cloned as an EcoRl/XmaI fragment into the unique EcoRl/XmaI sites of pSB2034. The resulting plasmid was confirmed to contain the SSL11 promoter region directly upstream of the vector-borne lux/gfp, thus creating a transcriptional fusion (pJLED1), by sequencing the promoter region and the insertion sites. Both pJLED1 and pSB2034 were recovered from E. coli and introduced into S. aureus Newman. Transfer of both plasmids was confirmed by PCR and digestion of extracted staphylococcal DNA, and by green fluorescence of the transformants.

GFP and Lux Expression Analysis

For quantification of bioluminescence in the absence of exogenous aldehyde, overnight cultures of S. aureus Newman harbouring either pSB2034 or pJLED1 were diluted 1/50 into medium containing the necessary antibiotics. Samples of each condition were prepared in triplicate and loaded into a 96-well microtiter plate (320 μl) and incubated at 37° C. without shaking in a Luminoskan luminometer (Thermo Electron Corp., Burlington, ON). Both OD₆₀₀ and luminescence were measured every hour for 48 hours and the luminometer was programmed to read each well for 10 s. Results were calculated as relative light units (RLUs) and the data were normalized by taking the maximum RLU detected divided by the corresponding maximum colony-forming unit (CFU) value for each condition. GFP was detected using fluorescence spectroscopy (Olympus BX-61 light microscope with a FITC filter). Fluorescent images were analyzed using the Pro-Image Plus program version 5.0.1 (Media Cybernetics). All media used to grow S. aureus Newman in the promoter expression analysis were diluted 1:1 in Milli-Q water. For the repression of an activated SSL11 promoter, 1/150 dilution of concentrated RC-14 supernatant (in Milli-Q water) was added to S. aureus Newman growing in BHI medium at 26 h. At the same time point, an equivalent volume of Milli-Q water was added to S. aureus Newman growing in BHI medium or L. reuteri RC-14 supernatant.

Screening for the Cell-cell Signalling Compound

Initially, 2 litres of L. reuteri RC-14 cell-free supernatant was lyophilized and resuspended in 100 mL of 100% methanol. The concentrated supernatant was then centrifuged (5000×g, 10 min, 4° C.), passed through Whatman No. 1 filter paper and then a 0.45 μm filter to remove particulate material. Rotary evaporation was used to concentrate the soluble portion of the supernatant to 1/100 of the volume of the original culture supernatant. A aliquot (400 μl) of concentrated supernatant was put across a P-10 gel-filtration column (BioRad Laboratories). Fractions were collected, and those testing positive for biological activity in the luminescence 96 well plate assay were dried, resuspended in water, and examined by high-performance liquid chromatography (HPLC) on an Agilent 1100 HPLC (Agilent Technologies, Mississauga, ON). Analytical reversed-phase HPLC was used for final purification of the cell-cell signalling molecule using a Zorbax 300SB-C8 (4.6×250 mm, 5 μm in diameter) column (Agilent). Solvent A consisted of trifluoroacetic acid (0.1%) in water and solvent B consisted of trifluoroacetic acid (0.085%) in acetonitrile and all solvents used were of HPLC grade (Fischer Scientific, Ottawa, ON). The chromatographic method used was as follows: flow rate of 1.5 mL/min, 2% B for 2 min, followed by gradient of 2% to 80% B over 30 min and a final step of 80% B for 2 min. Fractions were collected off the HPLC C8 column and tested in the luminescence 96 well plate assay. In order to determine if the repression of both the SSL11promoter and the staphylococcal P3 promoter was dependent on the same compound from the L. reuteri RC-14 supernatant, we performed the 96-well luminescence assay with S. aureus Newman containing either pJLED1, or pSB2034, grown in either BHI medium, L. reuteri RC-14 supernatant or a fraction collected off the HPLC that had previously tested positive for the ability to repress the SSL11 promoter.

Cloning, Expression and Purificadion of ssl11

585 base pairs of the ssl11 gene lacking DNA encoding the N-terminal signal peptide were PCR amplified using primers ssl11 (forward) and ssl11 (reverse) and cloned into the multiple-cloning site of pET28a. This construct encodes SSL11 tagged at the N-terminus with His₆. The ligation mixture was transformed into E. coli DH5a and the resulting clone was confirmed by sequencing. E. coli BL21 (λDE3) was then transformed with the pET28::ssl11 clone for protein over expression and the resulting clone was confirmed by sequencing and expression analysis.

For gene expression, E. coli BL21 (λDE3) harbouring recombinant pET28::ssl11 was grown overnight to an approximate OD₆₀₀ of 0.8, diluted 1:50 in fresh broth and incubated for a further 1 h at 37° C. Gene expression was induced with 1 mM isopropyl-β-D-thiogalactopyranoside (IPTG) for 3 h at 37° C. Cells were harvested by centrifugation at 7500×g for 10 min and then resuspended in 400 μl of Bug Buster protein extraction reagent (Novagen) supplemented with 10 U of DNAse (Sigma) and 1 mM of MgCl₂ according to manufacturer's directions. Recombinant proteins were purified using Ni²⁺affinity chromatography as specified by the manufacturer (Qiagen Ltd.). The N-terminal His₆-tag was removed by cleavage with 1 U of Thrombin (Sigma) per mg of recombinant protein (16 h at 21° C.). The recombinant protein was purified from the His₆-tag and thrombin by anion exchange chromatography using a DEAE column pre-equilibrated in 20 mM Tris-HCI buffer pH 7.4 (BioRad Laboratories). Elution of the proteins was achieved by increasing concentrations of KCl, and SSL11 was dialyzed against PBS (4° C., 12 h) to remove excess salt. Protein purity was verified by SDS-PAGE.

Peripheral Blood Lymphocyte Proliferation Assay

The ability of purified recombinant SSL11 to stimulate human mononuclear cells to proliferate was assessed using gradient purified human peripheral blood mononuclear cells (PBMCs) stimulated in vitro in 96-well microtiter lates (2□10⁵ cells/well) with serial 1:10 dilutions (in quadruplicate) of purified toxic shock syndrome toxin-1 (TSST-1) as a positive control (McCormick et al., J Immunol 171: 1385-1392, 2003) or SSL11. RPMI medium (Gibco, Invitrogen Corp.,) supplemented with 10% fetal calf serum (Sigma), 100 μg/mL streptomycin (Sigma), 100 units/mL penicillin (Sigma), and 1% L-glutamine (Sigma) was used as the culture medium and cells were incubated in 7% CO₂ at 37° C. Cells were pulsed with 1 μCi/well of [³H]Thymidine after 72 h and after another 18 h cells were harvested on fiberglass filters and [³H]Thymidine incorporation was assessed on a 1450 Microbeta liquid scintillation counter (Wallac, Turku, Finland). Background was considered as counts from cells not treated with toxin.

Serum Antibody Titre and Western Blot Analysis

Serum titre of IgG class antibody raised to staphylococcal antigens was measured by ELISA, according to a method described previously (Sambrook and Russell, 2001). The antigens were either 1 μg of staphylococcal cell wall-associated proteins (control) or 1 μg of SSL11. Serial dilutions of sera (20- to 40 000-fold) from five patients who previously had acute S. aureus bacteraemia were used as primary antibodies. Secondary goat anti-human IgG antibody (Sigma-Aldrich) was used at a 2000 fold dilution. Western blot analysis (Sambrook and Russell, 2001) was performed using the same human serum samples as primary antibodies at a 500-fold dilution, and the secondary antibody used was goat anti-human antibody conjugated to horseradish peroxidase (Sigma-Aldrich) at a 1000-fold dilution.

Production of Anti-SSL11 Antisera

Polyclonal antisera to His₆-SSL11 were generated in a New Zealand white rabbit at the Animal Care and Veterinary Services facility at the University of Western Ontario. In brief, 150 μg of protein emulsified in Freuds Complete Adjuvant was injected subcutaneously and subsequent boosts were done with 75 μg of protein emulsified with Freuds Incomplete Adjuvant on days 14, 21, 28, 35, 65 and day 96. On day 96 the antisera were recovered.

Statistical Analysis

Statistical comparisons between independent means were made by the Student's t test assuming unequal variances. Significant differences (P-values<0.05) between independent means were determined.

Results

2-DE of S. aureus Newman Cell Wall-associated Proteins in Response to Growth with L. reuteri RC-14

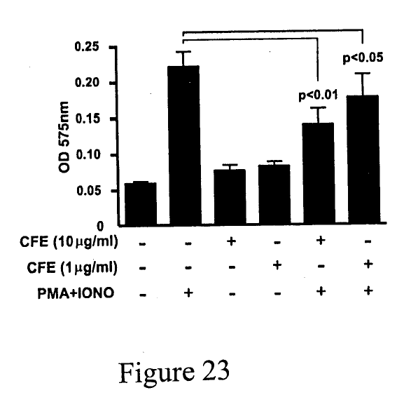

The goal of this study was to characterize cell-cell communication events between S. aureus and L. reuteri RC-14 that may help to mechanistically explain the ability of L. reuteri RC-14 to inhibit S. aureus infection (Gan et al., J Infect Dis 185: 1369-1372, 2002). To analyse protein expression patterns from S. aureus in response to growth with L. reuteri RC-14, the present Example focused on a sub-proteome enriched for surface proteins of S. aureus. This was achieved by extraction of cell wall-associated proteins. The protein expression profiles of the cell wall-associated preparations analyzed by 2-DE were very similar between all gels, with the total number of spots varying between 118 and 130, and the number of spots per gel for a given condition remaining constant (FIG. 1.). Specifically, when S. aureus Newman was co-cultured with BHI, L. reuteri RC-14 or L. rhamnosus GR-1, the gels had 122, 130 and 118 detectable spots, respectively. In each of these co-culture conditions, there were no significant differences detected between the OD₆₀₀ values of S. aureus Newman when samples were taken during late exponential phase (results not shown). Each condition was repeated a minimum of 3 times, with the gels shown in FIG. 23 being a representative experiment.

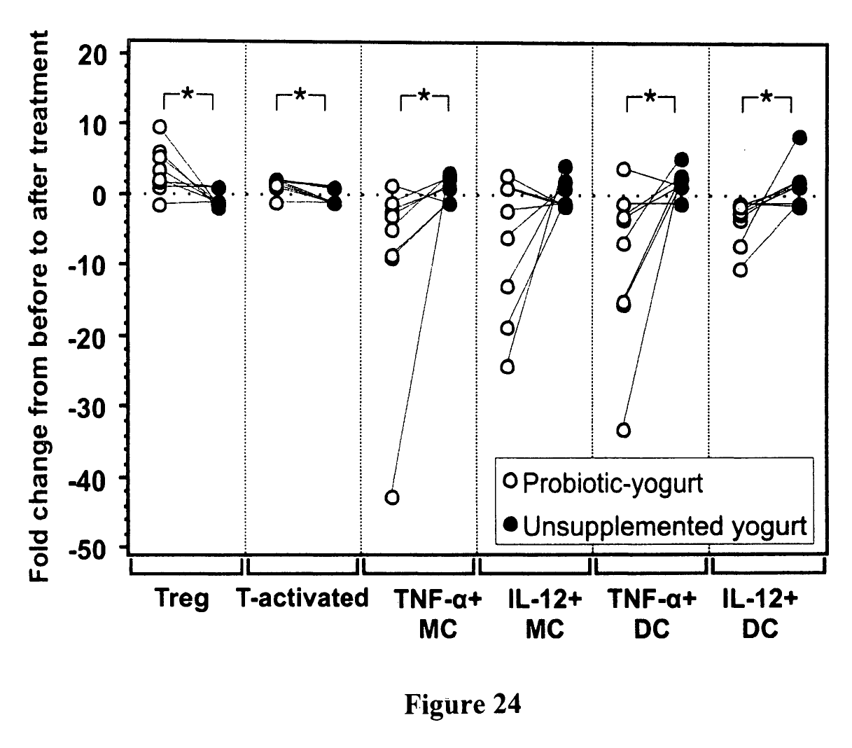

Only proteins whose fold of expression change was greater than 2 were further analyzed. To identify specific staphylococcal proteins that either decreased or increased in expression in response to co-culture with L. reuteri RC-14, seven of the spots in the preparative gels were selected for further analysis (FIG. 23A and FIG. 23B). Protein identification was done by MALDI-TOF and MS/MS analysis of tryptic peptide digests. Using the completed S. aureus genome sequences (Baba et al., Lancet 359: 1819-1827, 2002; Gill et al., J Bacteriol 187: 2426-2438, 2005; Holden et al., Natl Acad Sci USA 101: 9786-9791, 2004; Kuroda et al., Lancet 357: 1225-1240, 2001), the 7 spots were identified. The 7 spots corresponded to 7 individual proteins. Properties of those, including putative functions, are listed in Table 8. These 7 proteins were identified in all completed S. aureus genomes published to date. One protein of interest (labelled 1, FIG. 23A) was identified which decreased considerably and, at times, disappeared completely when S. aureus was co-cultured with L. reuteri RC-14. This protein had an approximate molecular mass of 24 kDa and an approximate isoelectric point (pI) of 5.9. The sequence tag from the MS analysis of this protein is 100% homologous to a predicted translated contig from the genome of S. aureus COL (locus SA0478) (FIG. 24A) (Gill et al., J. Bacteriol 187: 2426-2438, 2005) which corresponds to a 675-bp open reading frame (ORF) encoding a predicted protein of 225 amino acids. Three allelic variants of the translated gene were found in other S. aureus strains (FIG. 24B). SSL11 belongs to the group of proteins known as the staphylococcal superantigen-like proteins (SSL proteins) (Lina et al., J Infect Dis 189: 2334-2336, 2004), which are thought to be important virulence factors in S. aureus (Al-Shangiti et al., Infect Immun 72: 4261-4270, 2004; Arcus et al., J Biol Chem 277: 32274-32281, 2002; Fitzgerald et al., Infect Immun 71: 2827-2838, 2003; Langley et al., J Immunol 174: 2926-2933, 2005).

Both the Staphylococcal SSL11Promoter and P3 Promoter are Repressed in the Presence of L. Reuteri RC-14 Supernatant

Based on the observation that a potential exotoxin-like protein decreased in expression in response to co-culture with L. reuteri RC-14, a dual gene reporter construct to study ssl11 gene expression was constructed. Based on the S. aureus COL sequence (Gill et al., J Bacteriol 187: 2426-2438, 2005), PCR primers P_{SSL11}::gfp/lux forward (SEQ ID NO: 18) and P_{SSL11}::gfp/lux reverse (SEQ ID NO: 19) were used to amplify the SSL11 promoter from S. aureus Newman. This region corresponds to a 385-bp region upstream of the ssl11 gene and immediately downstream of a putative type-IC restriction-modification system (FIG. 24A). The nucleotide sequence of this region was 100% homologous to the same region in S. aureus strains COL (Gill et al., J Bacteriol 187: 2426-2438, 2005), Mu50 (Kuroda et al., Lancet 357: 1225-1240, 2001), N315 (Kuroda et al., Lancet 357: 1225-1240, 2001), and MRSA252 (Holden et al., Proc Natl Acad Sci USA 101: 9786-9791, 2004). Interestingly, in S. aureus strains MSSA476 (Holden et al., Proc Natl Acad Sci USA 101: 9786-9791, 2004) and MW2 (Baba et al., Lancet 359: 1819-1827, 2002), the first 79 base pairs of this sequence showed little to no homology to the Newman/COL sequence (FIG. 24A). This promoter region replaced the staphylococcal P3 promoter in the Gram-positive expression vector pSB2034, creating the gene reporter construct pJLED1. As the expression of most exoproteins in S. aureus is regulated by the two-component agr signalling pathway, the expression by the P3 promoter (pSB2034 construct) was also monitored. P3 is one promoter of the agr system whose gene product, RNAIII, is the effector molecule of the agr system (Novick et al., Embo J 12: 3967-3975, 1993). Both reporter constructs, pJLED1 and pSB2034, were introduced separately into S. aureus Newman. S. aureus harbouring these gene reporter constructs were grown in two separate microtiter plates, to monitor hourly luminescence and OD₆₀₀ readings, respectively. For optimum activation of both the SSL11 and P3 promoter, S. aureus was grown in BHI media diluted 1:1 with Milli-Q water. As a result of this, all further media used in promoter expression experiments were diluted 1:1 with Milli-Q water. These strains were grown in a 96 well plate assay in the presence or absence L. reuteri RC-14 supernatant (FIG. 25A). The results of these experiments indicate that both P_{SSL11} and P3 are activated in stationary phase when S. aureus Newman is grown in BHI with a peak of luminescence detected at approximately 30 h of growth (FIG. 25A) and that both P_{SSL11} and P3 were repressed when S. aureus was grown in the presence of L. reuteri RC-14 supernatant. The data obtained from the luciferase experiments were also confirmed by monitoring S. aureus grown in BHI or L. reuteri RC-14 supernatant for the expression of GFP (results not shown). Importantly, growth of S. aureus is slightly inhibited when grown in L. reuteri RC-14 supernatant when compared to growth in BHI (an effect likely due to a decrease in nutrients available in the spent supernatant, as S. aureus grows equally well in BHI supplemented with concentrated L. reuteri RC-14 supernatant). To compensate for the decrease in growth observed from S. aureus in L. reuteri RC-14 supernatant, the maximum RLU detected from each condition was measured and the result was then divided by the corresponding maximum colony forming unit (CFU) for a variety of different conditions (FIG. 25B). Based upon normalized data, it was determined that the SSL11 promoter remains repressed in the presence of the L. reuteri RC-14 supernatant and that the observed effect was not due to a change in pH or to the presence of lactic acid or hydrogen peroxide in the L. reuteri RC-14 supernatant (FIG. 25B). It was also determined that the repression of the SSL11 promoter could be diluted out by adding decreasing amounts of L. reuteri RC-14 supernatant to S. aureus Newman (pJLED1) growing in BHI medium. Finally, it was determined that the ability of the L. reuteri RC-14 supernatant to repress SSL11 promoter activation was insensitive to protease activity (pronase, proteinase K, trypsin and a cocktail of all 3) or to heat (FIG. 25B).

Since L. reuteri RC-14 supernatant repressed transcription from the SSL11 promoter, spent culture supernatant was also tested to determine if it could repress an activated SSL11 promoter. To accomplish this, a 1/150 dilution of concentrated supernatant was added to S. aureus Newman (pJLED1) growing in BHI medium at the 26 h time point. The assay was performed in triplicate, and an equivalent volume of water was added to the same strain of S. aureus growing in either BHI medium or L. reuteri RC-14 supernatant. A repression of the SSL11 promoter was observed within 1 hour following addition of concentrated supernatant indicating that L. reuteri RC-14 supernatant is able to repress an activated SSL11 promoter. No repression of the SSL11 promoter was observed upon addition of an equivalent volume of water to S. aureus growing in BHI.

Expression and Repression of the SSL11 Promoter is Independent of the agr System in S. aureus

The expression of many virulence factors in S. aureus is controlled by agr, a quorum sensing system that upregulates the expression of many secreted proteins upon entering late-exponential phase, and at the same time represses the expression of many cell wall-associated proteins. The present study was interested in determining if the decrease in ssl11 expression observed when S. aureus Newman was grown in the presence of L. reuteri RC-14, or L. reuteri RC-14 supernatant, was a direct consequence of the decrease in activation of the P3 promoter. In order to further analyze this, pJLED1 was transformed into another S. aureus wild-type strain, RN6390, and its isogenic agr mutant, RN6911. These strains exhibited the expected characteristic phenotypes when plated on sheep blood agar plates. RLUs were detected from each of these strains when they were grown in BHI medium or L. reuteri RC-14 supernatant for a 48 h time period. SSL11 promoter activation was observed when each of these strains were grown in control BHI medium, and repression of the SSL11 promoter was observed when each of these strains were grown in L. reuteri RC-14 supernatant, indicating that both expression, and repression, of the SSL11 promoter is independent of P3 and the agr system of S. aureus.

Fractionated RC-14 Supernatants Have Differential Effects on P_{SSL11} and P3 Repression.

In order to begin characterization of the molecule(s) mediating the repression of the SSL11 and agr promoters, RC-14 supernatant was methanol extracted and subjected to size exclusion chromatography followed by reversed phase HPLC. Fractions were screened for activity using S. aureus Newman harbouring pJLED 1, and luminescence was used as a measure of SSL11and agr promoter activity. It was observed that an active HPLC fraction was able to completely inhibit activation of P_{SSL11}, but had only moderate inhibitory activity against P3.

SSL11 Does Not Stimulate Peripheral Blood Lymphocytes

The SSL11 gene predicts a protein with a classical 30 amino acid signal sequence, with the mature protein of 195 amino acids. To investigate a possible function of SSL11, ssl11 lacking the signal peptide from S. aureus Newman was cloned into an E. coli expression vector for expression of ssl11 as a His-tagged protein. Sequencing of the ssl11 gene from S. aureus Newman revealed it was 100% identical to ssl11 in S. aureus strains COL (Gill et al., J Bacteriol 187: 2426-2438, 2005) and NCTC 8325 (www.genome.ou.edu/staph.htmL) (FIG. 24B). SSL11 was purified and used in a standard peripheral blood lymphocyte proliferation assay to confirm previous reports that SSL11 isolated from a different S. aureus strain was not mitogenic, and therefore does not fit the criteria for a superantigen (Arcus et al., J Biol Chem 277: 32274-32281, 2002). TSST-1 was used as a positive control, and reaches maximum stimulation at 50 ng/mL in the experiment performed, and has half maximal proliferation at approximately 500 pg/mL. Confirming previous findings (Arcus et al., J Biol Chem 277: 32274-32281, 2002), at all concentrations tested, SSL11 was not able to induce T cell proliferation.

Sera from Patients Infected with S. aureus Contain Antibodies to SSL11

As SSL11 can be a potential bacterial virulence factor, the present study tested if it is immunogenic to humans. In order to determine this, five sera from patients with S. aureus bacteremia were characterized for the presence of SSL11 reactive antibodies by ELISA and Western blot analysis using cell wall-associated staphylococcal proteins, or pure SSL11 as the antigen. The results from both the Western blot analysis and ELISA analysis demonstrated that all patients contained anti-staphylococcal cell wall-associated protein antibodies and anti-SSL11 antibodies, likely indicating that SSL11 is both immunogenic and expressed during the course of an in vivo infection.

TABLE 7
Bacterial strains, plasmids and oligonucleotides used in this study^{a}

| Designation | Description | Source or reference |
|---|---|---|
| Strains | | |
| S. aureus | | |
| Newman | Wild type strain | (Dajcs et al., 2002) |
| RN4220 | Restriction-deficient derivative of 8325-4 | (Kreiswirth et al., 1983) |
| | accepts foreign DNA, (rₖ⁻mₖ⁺) | |
| RN6390 | Prophage-cured wild type strain | (Novick et al., 1993) |
| RN6911 | agr null mutant of RN6390 containing a 3-kb | (Peng et al., 1988) |
| | fragment with tet^{r} marker in place of the 3.4-kb | |
| | agr ClaI-to-HinCII fragment | |
| MSSA476 | Invasive community acquired methicillin sensitive | (Holden et al., 2004) |
| | strain | |
| E. coli | | |
| DH5α | F⁻ φ80 dlacZ ΔM15 Δ(lacZYA-argF) U169 endA1 | Gibco-BRL |
| | recA1 hsdR17 (rₖ⁻mₖ⁺) deoR | |
| BL21 (λDE3) | F⁻ ompT [Ion] hsdSB (an E. coli B strain) with the | Novagen |
| | λDE3 prophage carrying the T7 RNA polymerase | |
| | gene | |
| Lactobacilli | | |
| L. reuteri RC-14 | Isolated from the urogenital tract of a healthy | G. Reid |
| | women | |
| L. rhamnosus GR-1 | Isolated from the urogenital tract of a healthy | G. Reid |
| | women | |
| Plasmids | | |
| pSB2034 | P3 promoter controlling expression of both | (Qazi et al., 2001) |
| | luxABCDE from Photorhabdus luminescens | |
| | and translationally enhanced gfp3 (red-shifted | |
| | gfp variant), Cm^{r}, Ap^{r} | |
| pJLED1 | SSL11 promoter amplified by PCR from S. | This study |
| | aureus Newman inserted into the EcoRI and | |
| | XmaI sites of pSB2034; Cm^{r}, Ap^{r} | |
| pET28a | E. coli expression vector; expression is under the | Novagen |
| | control of the T7 promoter; Km^{r} | |
| pET28a::ssl11 | ssl11 amplified by PCR from S. aureus Newman | This study |
| | inserted into XhoI and NdeI sites of pET28a; Km^{r} | |
| Primers^{b} | | |
| PSSL11::gfp/lux (forward) | CACCGAATTCTAACTTTGATAAATACATAG | (SEQ ID NO: 18) |
| PSSL11::gfp/lux (reverse) | CATCAACCCCGGGATTCTATGCTCCCAATT | (SEQ ID NO: 19) |
| ssl11 (forward) | CACTCAACATATGAGTACATTAGAGGTTAGATC | (SEQ ID NO: 20) |
| ssl11 (reverse) | CACCCTCGAGGCTCCCTCGAATAATTTTA | (SEQ ID NO: 21) |

^{a}Abbreviations: Ap^{r}, Cm^{r}, Tet^{r}, Km^{r}, resistance to ampicillin, chloramphenicol, tetracycline and kanamycin respectively.
^{b}Restriction sites for subsequent cloning of the PCR products are underlined.

TABLE 8
Characteristics of proteins identified in 2-DE

| | | | | | pI | |
|---|---|---|---|---|---|---|
| | | Protein (S. aureus Mu50) | | Mass | Measured/ | |
| Spot | Fold Change | (accession number) | Sequence Tag | (kDa) | Theoretical | Coverage |
| 1 | −5.6 | SSL11 | SGNTASIGGITK | 24/22.3 | 5.9/6.0 | 64 |
| | | (NP_370957) | (SEQ ID NO: 22) | | | |
| 2 | −2.1 | Cysteine synthase | TIDAFLAGVGTGGTLSGVGK | 34/33 | 5.4/5.5 | 21.29 |
| | | (NP_371037) | (SEQ ID NO: 23) | | | |
| 3 | +2.1 | Superoxide dismutase | LNAAVEGTDLESKSIEEIVANL | 25/22.7 | 5.1/5.2 | 17.08 |
| | | (NP_372077) | DSVPANIQTAVR (SEQ ID NO: 24) | | | |
| 4 | −2.6 | Hypothetical protein | LTLQVVSIDEQGK | 24/22.3 | 5.4/5.3 | 16.58 |
| | | (NP_372378) | (SEQ ID NO: 25) | | | |
| 5 | +2.1 | 30S ribosomal protein | AGQFYINQR | 34/29.1 | 5.6/5.4 | 7.84 |
| | | (NP_371780) | (SEQ ID NO: 26) | | | |
| 6 | +8.6 | Formyltetrahydrofolate synthetase | IVTEIYGGSK | 60/59.8 | 5.8/5.7 | 4.8 |
| | | (NP_372256) | (SEQ ID NO: 27) | | | |
| 7 | −2.4 | ABC transporter (ATP Binding protein) | YLNEGFSGGEK | 28/29.1 | 4.8/4.7 | 7.9 |
| | | (NP_371366) | (SEQ ID NO: 28) | | | |

### EXAMPLE 11

### Materials and Methods

Subjects

The present study population comprised of 20 subjects with inflammatory bowel disease (IBD) and 20 age-matched healthy controls with no known or suspected intestinal abnormalities. The mean age ±SD of IBD patients was 44±11.7 (range 26-63) years and that of controls 51±6.4 (38-61) years. Of the IBD patients 15 had Crohn's disease, 5 ulcerative colitis and all had subjective symptoms, including liquid or very soft stools and/or abdominal pain, indicative of active IBD. To reduce patient to patient variability, all the subjects were women. Exclusion criteria included pregnancy, use of antibiotics, lactose intolerance and premature termination of the study (only 3/23 healthy subjects were excluded due to inability to comply with the study protocol).

All subjects were asked to continue with their habitual diet but to refrain from taking any other yogurt or probiotic supplements two weeks before and during the study. The patient group did not alter any ongoing medication being given for their IBD. Informed consent was obtained from all subjects and the study was approved by the Review Board for Health Sciences Research involving Human Subjects, at the University of Western Ontario, London, Ontario, Canada.

Design

In this open-labeled study, all subjects consumed 125 g of probiotic-yogurt per day for 30 days. The researchers were blinded regarding the study groups. To rule out the influence of yogurt alone, the treatment regimen was repeated in an exploratory study with unsupplemented yogurt with a subpopulation of the same IBD patients (n=8; 6 with Crohn's disease, 2 with ulcerative colitis) after a washout period of six months.

The main outcome parameters measured were changes in the prevalence of putative Treg-cells (CD4+CD25 high) and TNF-α and IL-12 producing monocytes and dendritic cells (DC) in peripheral blood (PB) during treatment. Secondary outcome measures included changes in the presence of T-cell surface activation markers, serum and fecal cytokine levels and ex vivo proliferative responses of PB mononuclear cells (PBMC). Individual stool and blood samples were collected before (day 0) and after (day 30) the treatment period. The patients were asked to note in a diary any changes in symptoms, including bloating, gas, abdominal pain and constipation/loose stools, throughout the study.

Preparation of Probiotic-yogurt

To prepare a probiotic-mother culture, dried Lactobacillus rhamnosus GR-1

(GR-1) and Lactobacillus fermentum RC-14 (RC-14) (Canadian Research and Development Centre for Probiotics, London, ON) were added to Man, Rogosa, and Sharpe broth (EM Science, Gibbstown, N.J.) at a rate of 1.5% and grown anaerobically at 37° C. overnight. Then a mixture of milk (1% fat), 0.33% yeast extract, and 0.4% inulin was autoclaved for 15 min, cooled to 37° C., and inoculated with the probiotic culture at a rate of 1% and incubated anaerobically at 37° C. overnight. To prepare probiotic-yogurt, a mixture with milk (1% fat) and 5% sugar was heat-treated at 87° C. for 30 min, cooled to 37° C., inoculated with 4% of the probiotic-mother culture and 2% of standard plain yogurt containing L. delbreukii subsp. bulgaricus and Streptococcus thermophilus, fermented at 37° C. for 6 h and stored at 4° C. After two days 11% strawberry flavoring (Sensient, Rexdale, ON) was added and the yogurts were packaged. Viable counts and quality assurance was tested at regular intervals. A new batch of yogurt was produced every two weeks to ensure consistency in viable counts of the probiotic bacteria, especially as those of RC-14 fell rapidly with time. After two weeks at 4° C. the total counts were consistently at 1×103 for RC-14 and 2×107 cfu/mL for GR-1. No contaminants were isolated at any time in the study.

Analysis of Intracellular Cytokine Production

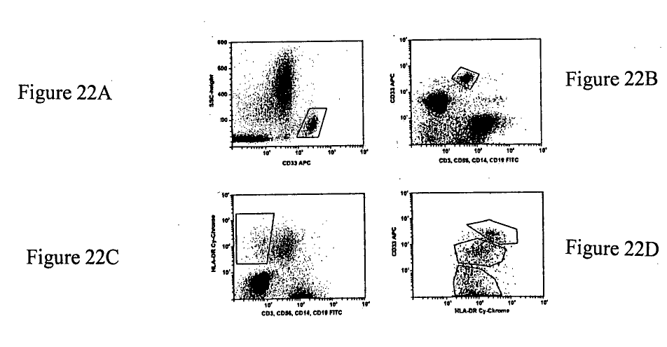

Intracellular cytokine detection was performed by flow cytometry as previously described with some modifications (8, 9). PB samples in lithium heparin were supplemented one to one with RPMI 1640 medium (Invitrogen, Burlington, ON), incubated at 37° C. in a 5% CO2 humidified atmosphere with Brefeldin A (10 μ/mL, Sigma, St. Louis, Mo.) in the presence or absence of: lipopolysaccharide (LPS, 100 ng/mL; from Escherichia coli, serotype 055:B5, Sigma) plus IFN-γ (100 Units/mL; R&D Systems, Inc., Minneapolis, Minn.) for stimulation (6 h) of cytokine production by monocytes and DC; ionomycin (1 μg/mL, Sigma) plus phorbol 12-myristate 13-acetate (PMA, 25 ng/mL, Sigma) for stimulation (4 h) of cytokine production by T-cells. For the identification of the whole DC population (MHC II+/Iineage-/CD33+/−), their highly and intermediately CD33-expressing myeloid (CD33 high, CD33intermed) and no or weakly CD33-expressing plasmocytoid (CD33-/low) subsets and monocytes (MHC II+/CD14+/CD33+), PB cells were then incubated for 15 min at room temperature (RT) with anti-HLA-DR-Cy-chrome, anti-CD33-allophycocyanin (APC) and each of the following fluorescein isothiocyanate (FITC)-labeled lineage marker antibody: anti-CD3, anti-CD19, anti-CD56 and anti-CD14 (BD Biosciences, San Diego, Calif.). Stained cells were washed with phosphate-buffer saline (PBS, pH 7.5) and centrifugation (5 min at 540 g), fixed, permeabilized, and stained with anti-TNF-α-phycoerythrin (PE, clone MAb11) and anti-IL12-PE (C11.5) using the Fix & Perm reagent (Caltag, Burlingame, Calif.) following manufacturer's instructions. T-cell cytokines were analyzed accordingly, but the cells were identified with anti-CD3-FITC and their cytokines detected with anti-IL-2-PE (clone MQ1-17H12), anti-IFN-γ-PE (B27), anti-IL-4-PE (8D4-8) and anti-IL-10-PE (JES3-19F1). Data acquisition was performed in two consecutive steps with a flow cytometer (FACSCalibur™, BD Biosciences). First, 30,000 events/test corresponding to the whole PB cellularity were collected for analysis of cytokines produced by T-cells and monocytes. Second, only events in a HLA-DR+/CD3−/CD19−/CD56−/CD14− live gate were stored and a minimum of 300,000 events from the total PB cellularity were acquired in order to obtain at least 1000 MHC II+/lineage-cells for the analysis of cytokines produced by DC subsets. CellQuest™ software (BD Biosciences) was used for data acquisition and analysis. Representative acquisition dot plots demonstrating the identification of monocytes and DC are presented in FIG. 22.

Analysis of T-cell Surface Markers

For the expression of early activation marker CD69 on T-cells, RPMI-diluted PB was incubated with or without PMA and ionomycin as described above whereas only unstimulated sample was used for Treg-cell analysis. The percentage of CD4+CD25+ Treg-cells are enriched within the 1-2% of PB CD4+ T-cells expressing high levels of CD25 while the population expressing lower levels of CD25 is thought to consist mainly activated effector T-cells (10). Thus, using flow cytometry the present study gated onismall lymphocytes and CD4+ T-cells were subdivided into bright (CD4+CD25 high/Treg) and intermediate (CD4+CD25+/activated T-cell) populations based on their CD25 expression. The stimulated and/or unstimulated samples (200 μL each) were stained with 3 μL of anti-CD3-FITC in combination with anti-CD69-PE or anti-CD4-FITC plus anti-CD25-PE (BD Biosciences) for 15 min at RT. Data was acquired with flow cytometer (30,000 events/test) and analyzed as described above.

Enzyme-linked Immuno-sorbent Assays

Fecal extracts were prepared by mixing 3 grams of stool with 3 mL of PBS followed by centrifugation (30-45 min at 20,000 g) at 4° C. and filtration of the supernatant through a 0.45 μm-pore-size filter (11). Serum samples and fecal extract aliquotes were stored at −70° C. until analyses. The levels of TNF-α, IL-12 and IL-10 were measured with BD OptEIA™ ELISA Sets (BD Biosciences) according to manufacturer's instructions.

Proliferation Assay

Cell-free extracts (CFE) of RC-14 and GR-1 were prepared from capsules containing 1×109 cfu of RC-14 and GR-1 (12, 13). The bacteria were washed twice and suspended in PBS (1 mL) and then bead beat with 300 mg of zirconium beads (0.1 mm) (3 min at 5000 rpm) using a mini-bead beater (Biospec Products, Bartlesville, Okla.). Particulates were removed by centrifugation (10 min at 12,000 g) and the protein concentration in the supernatants (CFE) determined with the BCA protein assay kit (Pierce, Rockford, Ill.) with bovine serum albumin as the protein standard. PBMC were isolated from PB in sodiumheparin by Ficoll-Hypaque (Pharmacia Biotech, Uppsala, Sweden) gradient centrifugation. PBMC (0.5×106/mL) were cultured in RPMI 1640 with 2 mM Lglutamine, penicillin (100 U/mL), streptomycin (100 μg/mL), and 10% fetal bovine serum supplemented with CFE in the presence or absence of ionomycin (100 ng/mL) plus PMA (100 ng/mL) for 4 days at 37° C. in a 5% CO2 humidified atmosphere. Cultured cells were then further incubated on 96-well plates (200 μL/well in triplicates) for 4 h at 37° C. with 20 μL of 3-(4,5-dimethylthiazol-2-yl)-2,5-diphenyltetrazolium bromide (MTT, Sigma) (2.5 mg/mL in PBS) per well. The plates were centrifuged (5 min at 500 g) and supernatants were removed. HCL (0.04 N) in isopropanol (100 μL) was added to each well and absorbance measured at 575 nm (reference wavelength 650 nm) with a microplate reader (Bio-Rad Model 550).

Statistics

Statistical analysis was performed with Graph Pad Prism®, version 4 (GraphPad, Software, Inc., San Diego, Calif.) and StatView® version 4.57 (Abacus Concepts Inc., Berkeley, Calif.) with the exception of Exact unconditional test for 2 by 2 tables, which was used for comparing frequency of symptoms before and after treatment (14). Changes in immunological measurements between two timepoints within a subject group were compared with paired two-tailed t test if the data was parametric with or without natural logarithmic transformation and by the Wilcoxon signed rank test if the data was nonparametric and non-transformable. Differences between subject groups were compared with unpaired two-tailed t test if the data was parametric and with Mann Whitney U test if the data was nonparametric and non-transformable. Correlations between two continuous variables were analysed by Spearman rank correlation test. P values<0.05 were considered statistically significant.

Results

Effect of Probiotic-yogurt Intake on T-cells

The percentage of CD4+CD25 high cells increased significantly following the treatment with probiotic-yogurt from the group mean (95% confidence interval, CI) of 0.84 (0.55-1.12)% to 1.25 (0.97-1.54)% (p=0.007). In controls the response was significantly different (p=0.03) with little increase from before, 0.69 (0.50-0.87)%, to after, 0.73 (0.59-0.87)% the treatment (p=0.09). Similarly, the change in the percentage of CD4+CD25+ T-cells was significantly different between the groups (p=0.01) with increase from 9.1 (7.2-1 1.0)% to 11.0 (9.5-13.1)% (p=0.003) in IBD patients and no change from before, 6.68 (5.78-7.59)%, to after, 6.47 (5.69-7.24)%, the treatment in controls (p=0.36).

In IBD patients, but not in controls, the treatment was followed by reduced percentage of CD3+ T-cells responding to polyclonal ex vivo stimulation by production of IL-2. In IBD patients the mean percentage of IL-2+CD3+ T-cells was 42.3 (95% CI 35.4-49.2)% before and 38.2 (32.2-44.2)% (p=0.03) after the treatment, whilst the o0 respective values for controls were 42.4 (36.7-48.0)% and 44.4 (39.3-49.4)% (p=0.50). The difference in the change between the groups was not significant (p=0.20). No other significant effects were observed in the intracellular cytokine production by CD3+T-cells. However, the percentage of stimulated T-cells expressing CD69 decreased in the IBD patients (p=0.02) but, again, not in the control group (p=0.77). This difference between the groups approached statistical significance (p=0.07).

Effect of Probiotic-yogurt Intake on Monocytes and DC

The basal proportion (before treatment) of monocytes and DC which produced TNF-α or IL-12 was higher in the IBD patients compared to controls with some differences reaching statistical significance (p<0.05; Table 9). The proportion of monocytes or DC populations in PB per se did not change following treatmentwith probiotic yogurt whereas significant decreases were observed in the percentages of unstimulated TNF-α and IL-12 producing monocytes and myeloid DC subsets in both IBD patients and controls as summarized in Table 9. In unstimulated and/or stimulated plasmocytoid DC subset the production of these cytokines was very low or undetectable with no significant changes during the treatment. Significant correlations were observed between the change in the proportion of Treg-cells (increase) following the treatment and the change (decrease) in the proportion of unstimulated TNF-α and IL-12 producing monocytes (p=−0.59, p=0.01 and p =−0.58, p=0.01, respectively) and DC (ρ=−0.53, p=0.02 and p=−0.61, p=0.008, respectively).

TABLE 9
The ex vivo intracellular production of TNF-á and IL-12 by unstimulated
and stimulated peripheral blood (PB) monocytes and dendritic cells (DC) from
IBD patients and controls before and after treatment with probiotic-yogurt.
Percentage of cells in total in PB (mean ± SE)/
Percentage of cytokine producing cells (mean ± SE)

| | IBD Patients (n = 20) | | Controls (n = 20) | |
|---|---|---|---|---|
| | Before | After | Before | After |
| Cell type/cytokine | treatment | treatment | treatment | treatment |
| Monocytes | 4.9 ± 0.4 | 4.4 ± 0.4 | 4.0 ± 0.3 | 3.9 ± 0.4 |
| TNF+ basal^{a} | 6.4 ± 2.4* | 1.6 ± 0.5† | 2.7 ± 0.4 | 1.5 ± 0.3‡ |
| TNF+ stimulated^{b} | 58.1 ± 4.7 | 49.7 ± 3.3† | 50.7 ± 4.2 | 49.6 ± 3.7 |
| IL-12+ basal | 3.4 ± 0.7 | 1.5 ± 0.3† | 2.1 ± 0.2 | 1.2 ± 0.2† |
| IL-12+ stimulated | 21.7 ± 2.5 | 16.3 ± 2.0† | 17.2 ± 2.5 | 14.2 ± 2.5 |
| Dendritic cells (all) | 0.7 ± 0.1 | 0.7 ± 0.1 | 0.8 ± 0.1 | 0.7 ± 0.1 |
| TNF+ basal | 5.9 ± 1.7* | 1.4 ± 0.3† | 2.2 ± 0.3 | 1.2 ± 0.2‡ |
| TNF+ stimulated | 35.9 ± 3.5 | 27.3 ± 2.0† | #26.8 ± 3.7 | 29.3 ± 2.4 |
| IL-12+ basal | 2.1 ± 0.5 | 1.1 ± 0.2† | 1.2 ± 0.2 | 0.8 ± 0.1 |
| IL-12+ stimulated | 15.5 ± 1.9 | 9.6 ± 1.4‡ | 11.5 ± 2.1 | 10.6 ± 2.4 |
| DC CD33^{high} | 0.4 ± 0.1 | 0.4 ± 0.1 | 0.4 ± 0.05 | 0.3 ± 0.05 |
| TNF+ basal | 7.8 ± 2.5 | 1.9 ± 0.5† | 5.0 ± 1.2 | 1.2 ± 0.2‡ |
| TNF+ stimulated | 46.5 ± 4.4* | 42.5 ± 3.5 | 33.4 ± 4.0 | 37.1 ± 3.7 |
| IL-12+ basal | 3.0 ± 0.8 | 1.4 ± 0.3 | 2.0 ± 0.4 | 1.0 ± 0.6‡ |
| IL-12+ stimulated | 22.6 ± 3.3 | 14.7 ± 2.1† | 15.8 ± 2.1 | 9.7 ± 1.1 |
| DC CD33^{intermed} | 0.2 ± 0.03 | 0.2 ± 0.03 | 0.3 ± 0.03 | 0.5 ± 0.03 |
| TNF+ basal | 5.4 ± 1.4* | 1.5 ± 0.4‡ | 2.2 ± 0.4 | 1.5 ± 0.3† |
| TNF+ stimulated | 24.8 ± 4.0 | 22.3 ± 3.9 | 25.1 ± 4.9 | 24.4 ± 3.4 |
| IL-12+ basal | 3.3 ± 1.0 | 1.3 ± 0.3‡ | 1.1 ± 0.4 | 0.6 ± 0.2 |
| IL-12+ stimulated | 11.9 ± 2.5 | 7.7 ± 1.7† | 7.4 ± 1.5 | 7.4 ± 1.5 |

SE = standard error;
^{a}unstimulated PB culture (6 h);
^{b}LPS + IFN-ã stimulated PB culture (6 h);
*Level before treatment significantly different (p < 0.05) from that of controls;
#Change significantly different (p < 0.05) between IBD patients and controls;
†Significant change during treatment at significance level of 5% (p < 0.05);
‡Significant change during treatment at significance level of 1% (p < 0.01)

Effect of Probiotic-yogurt Intake on Serum and Stool Cytokines

The serum IL-12 concentration decreased significantly in both IBD patients and controls following the intake of probiotic-yogurt, the group mean (95% CI) decreasing from 51.6 (38.4-64.8) to 44.9 (34.5-55.4) pg/mL (p=0.02) in IBD patients and from 50.1 (41.5-58.8) to 46.1 (38.9-53.3) pg/mL in controls (p=0.03).

The levels of TNF-α and IL-10 were variable in IBD patients and no significant changes were observed. In controls, the serum levels of TNF-α decreased from group mean (95% CI) 7.6 (4.7-10.5) to 5.6 (3.4-7.8) pg/mL (p=0.002) while the fecal levels increased from 9.3 (3.6-15.0) to 14.2 (5.6-22.9) pg/mL (p=0.006) after treatment.

Patient Diaries

Analysis of patient diaries revealed two findings. One of twenty IBD patients reported excess intestinal gas at the time of recruitment and six at the end of the treatment period (p=0.02), whilst one of twenty reported subjectively low abdominal pain at the recruitment and six at the end of the treatment period (p=0.02). These latter six patients had significantly lower mean (95% CI) fecal concentration of IL-12, 9.1 (0.65-17.5) than the rest of the IBD patients (n=14), 13.0 (8.9-17.0) pg/mL (p=0.04) at end of the treatment period. No other significant changes or correlations to immunological variables were noted regarding the subjective symptoms.

In vitro Proliferative Responses of PBMC to CFE of RC-14 and GR-1

Addition of RC-14/GR-1 CFE to PBMC cultures induced only a marginal increase in proliferation compared to unstimulated PBMC from healthy controls whereas it appeared to inhibit the PMA+ionomycin induced proliferation (FIG. 23). Similar results were seen with PBMC from IBD patients and controls before and after consumption of probiotic yogurt.

Immunomodulatory Properties of Unsupplemented Yogurt

In the follow-up of 8 IBD patients no significant changes were observed in the percentage of Treg-cells, activated T-cells or TNF-α/IL-12 producing monocytes or DC following the 30-day intake of unsupplemented yogurt. These lack of changes were contrary to the significant changes that followed the intake of probiotic-yogurt as indicated in FIG. 24.

What is claimed is:

1. A method for altering the virulence or infectivity of pathogens in a mammal infected by the pathogens, comprising administering to said mammal a therapeutically effective amount of at least one signal molecule produced by non-pathogenic microorganisms so that the virulence of said pathogens is reduced.

2. A composition for altering the virulence or infectivity of a pathogen, wherein said composition comprises at least one signal molecule produced by non-pathogenic microorganisms so that the virulence of said pathogen is reduced.

3. The method of claim 1, wherein said non-pathogenic microorganism is Lactobacillus.

4. The composition of claim 2, wherein said non-pathogenic microorganism is Lactobacillus.

5. The method of claim 3, further comprising Bifidobacterium.

6. The method of claim 1, wherein said signal molecule is a protein or peptide molecule.

7. The method of claim 1, wherein said pathogens are Gram positive or Gram negative bacteria.

8. The method of claim 1, wherein said pathogens are pathogenic microorganisms selected from the group consisting of S. aureus, Enterococcus, Streptococcus, Staphylococcus, Clostridium, Shigella, Salmonella, E. coli , Prevotella, Gardnerella, Klebsiella, Pseudomonas, Campylobacter, Candida, Proteus, Burkholderia, Mycobacterium, Helicobacter, Bacteroides, Vibrio, Listeria, Yersinia, Chlamydia, Meningococcus, and Neisseria.

9. The composition of claim 4, wherein said Lactobacillus is selected from the group consisting of L. rhamnosus, L. casei, L. acidophilus, L. fermentum, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, L. iners and L. bifidum.

10. The composition of claim 4, wherein said Lactobacillus is selected from the group consisting of L. fermentum RC-14, L. reuteri RC-14, L. rhamnosus GR-1, Lactobacillus B-54, and L. jensenii PC1.

11. A method for the treatment or prevention of infections, comprising the administration of a therapeutically effective amount of at least one non-pathogenic microorganism in the form of viable cell or at least one signal molecule produced by said non-pathogenic microorganism.

12. The method of claim 11, wherein said infections are caused by pathogenic microorganisms.

13. The method of claim 11, wherein said infections are caused by biofilms.

14. A method for reducing the risk of infections associated with medical devices, comprising delivery of at least one signal molecule produced by non-pathogenic microorganisms to said medical devices or to sites surrounding said medical devices.

15. The method of claim 14, wherein said devices are selected from the group consisting of catheters, lines, stents, tubes, bags, valves, implants, and instruments.

16. A pharmaceutical composition suitable for treating or preventing infections in mammals, comprising a therapeutically effective amount of one or more of the signal molecules produced by non-pathogenic microorganisms and an acceptable carrier.

17. The pharmaceutical composition of claim 16 wherein said signal molecule is a protein or peptide molecule isolated from the group consisting of L. rhamnosus, L. casei, L. acidophilus, L. fermentum, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosae, L. iners, and L. bifidum.

18. The pharmaceutical composition of claim 16, wherein said carrier is a pharmaceutical carrier or natural foods.

19. The pharmaceutical composition of claim 16, wherein said carrier is selected from the group consisting of diapers, sanitary pads or feminine pads, feminine tampons, facial creams, wound dressings and oral products.

20. A method of reducing the symptoms and signs of infection in a mammal caused by pathogenic microorganisms, comprising administering to said mammal a therapeutically effective amount of at least one signal molecule produced by non-pathogenic microorganisms.

21. The method of claim 20, wherein the symptom is inflammatory bowel disease.


SEQUENCE LISTING (33 sequences)

SEQ ID NO: 1 - DNA, 20 bp, Artificial Sequence
  oligonucleotide
taactttgataaatacatag

SEQ ID NO: 2 - DNA, 19 bp, Artificial Sequence
  oligonucleotide
ttaaaccctcgtatcttaa

SEQ ID NO: 3 - protein, 12 aa, Staphylococcus aureus
SGNTASIGGITK

SEQ ID NO: 4 - protein, 20 aa, Staphylococcus aureus
TIDAFLAGVGTGGTLSGVGK

SEQ ID NO: 5 - protein, 34 aa, Staphylococcus aureus
LNAAVEGTDLESKSIEEIVANLDSVPANIQTAVR

SEQ ID NO: 6 - protein, 13 aa, Staphylococcus aureus
LTLQVVSIDEQGK

SEQ ID NO: 7 - protein, 9 aa, Staphylococcus aureus
AGQFYINQR

SEQ ID NO: 8 - protein, 10 aa, Staphylococcus aureus
IVTEIYGGSK

SEQ ID NO: 9 - protein, 11 aa, Bacillus halodurans
YLNEGFSGGEK

SEQ ID NO: 10 - protein, 12 aa, Staphylococcus aureus
SGNTASIGGITK

SEQ ID NO: 11 - protein, 20 aa, Staphylococcus aureus
TIDAFLAGVGTGGTLSGVGK

SEQ ID NO: 12 - protein, 34 aa, Staphylococcus aureus; identical to SEQ ID NO: 5

SEQ ID NO: 13 - protein, 13 aa, Staphylococcus aureus
LTLQVVSIDEQGK

SEQ ID NO: 14 - protein, 9 aa, Staphylococcus aureus
AGQFYINQR

SEQ ID NO: 15 - protein, 10 aa, Staphylococcus aureus
IVTEIYGGSK

SEQ ID NO: 16 - protein, 11 aa, Bacillus halodurans
YLNEGFSGGEK

SEQ ID NO: 17 - DNA, 30 bp, Artificial Sequence
  Synthetic oligonucleotide
caccgaattctaactttgataaatacatag

SEQ ID NO: 18 - DNA, 30 bp, Artificial Sequence
  Synthetic oligonucleotide
catcaaccccgggattctatgctcccaatt

SEQ ID NO: 19 - DNA, 33 bp, Artificial Sequence
  Synthetic oligonucleotide
cactcaacatatgagtacattagaggttagatc

SEQ ID NO: 20 - DNA, 29 bp, Artificial Sequence
  Synthetic oligonucleotide
caccctcgaggctccctcgaataatttta

SEQ ID NO: 21 - protein, 12 aa, Staphylococcus aureus
SGNTASIGGITK

SEQ ID NO: 22 - protein, 20 aa, Staphylococcus aureus
TIDAFLAGVGTGGTLSGVGK

SEQ ID NO: 23 - protein, 22 aa, Staphylococcus aureus
LNAAVEGTDLESKSIEEIVANL

SEQ ID NO: 24 - protein, 13 aa, Staphylococcus aureus
LTLQVVSIDEQGK

SEQ ID NO: 25 - protein, 9 aa, Staphylococcus aureus
AGQFYINQR

SEQ ID NO: 26 - protein, 10 aa, Staphylococcus aureus
IVTEIYGGSK

SEQ ID NO: 27 - protein, 11 aa, Staphylococcus aureus
YLNEGFSGGEK

SEQ ID NO: 28 - DNA, 400 bp, Staphylococcus aureus
taactttgataaatacatagattgcataagaataaaatttgtataatttaacataaaagttgtaaaagtaaagtgaattaaaaacgaacattaaatttaggcactgtgaaagcgcagtgtcttttttgtgtcgaaattgtgtacagaataagtagttaaataaagattaagttgagataaagtgttattcgtaaataaaagagagtagatcgataggaattgaatgatattagttaactatttattaaattacttaataatgattaatttttagttaaagtaagtttaatgtgaagcacgaccattgctcattataatgaatgaggattgttcgtattgcgtaatagaataaatcaaatagactaaaaattgggagcatagaattatgaaattaaaaaat

SEQ ID NO: 29 - protein, 5 aa, Staphylococcus aureus
MKLKN

SEQ ID NO: 30 - protein, 225 aa, Staphylococcus aureus
MKLKNIAKASLALGILTIGMITTTAQPVKASTLEVRSQATQDLSEYYNRPFFEYTNQSGYKEEGKVTFTPNYQLIDVTLTGNEKQNFGEDISNVDIFVVRENSDRSGNTASIGGITKTNGSNYIDKVKDVNLIITKNIDSVTSTSTSSTYTINKEEISLKELDFKLRKHLIDKHNLYKTEPKDSKIRITMKDGGFYTFELNKKLQTHRMGDVIDGRNIEKIEVNL

SEQ ID NO: 31 - protein, 227 aa, Staphylococcus aureus
MKLKNIAKASLALGILTTGMITTTAQPVKASTLEVRSQATQDLSEYYKGRGFELTNVTGYKYGNKVTFIDNSQQIDVTLTGNEKLTVKDDDEVSNVDVFVVREGSDKSAITTSIGGITKTNGTQHKDTVQNVNLSVSKSTGQHTTSVTSEYYSIYKEEISLKELDFKLRKHLIDKHDLYKTEPKDSKIRITMKNGGYYTFELNKKLQPHRMGDTIDSRNIEKIEVNL

SEQ ID NO: 32 - protein, 230 aa, Staphylococcus aureus
MKLKNIAKASLALGILTTGMITTTAQPVKAIEQSRLSVTSKDTQELKKYYSGTGYNFQNVSGYREGNKMNIIDGPQLNVVTLLGTDKERFKDDEDYEGLDVFVVREGSGKHADNISIGGITKTNKNQYKDPVQNVNLLTSKSNGQNTASVTSEYYSINKEEISLKELDFKLRKQLIDKHDLYKTEPKDSKIKVSMKNGGYYTFELNKKLQPHRMGDTIDSRNIKKIEVNL

SEQ ID NO: 33 - protein, 232 aa, Staphylococcus aureus
MKLKNIAKASLALGILTTGMITTTAQPVKASEQSRLSVTSNDTQELKKYYSGTGYNFQNVSGYREKDKMNIIDGTQLNVVTLLGTDKERFKDYDYDYEGLDVFVVREGSGKQAENISIGGITKTNKNDYKDFVNNVGLEITKPTGHNTATRQAETYRINKEEISLKELDFKLRKHLIENHELYKTEPKDGKIRITMKGGGYYTFELNKKLQPHRMGDVIDGRNIEKIEVDLY